(19) United States
(12) Patent Application Publication
(10) Pub. No.: US 2025/0241636 A1
(43) Pub. Date: Jul. 31, 2025

(54) METHOD FOR IMPLANT INSERTION

(71) Applicant: Surgical Fusion Technologies GmbH, Schlieren (CH)

(72) Inventors: Andrea MÜLLER, Winterthur (CH); Michele VANONI, Zürich (CH); Jörg MAYER, Niederlenz (CH)

(21) Appl. No.: 18/782,545

(22) Filed: Jul. 24, 2024

Related U.S. Application Data
(63) Continuation of application No. 18/605,571, filed on Mar. 14, 2024.
(60) Provisional application No. 63/626,292, filed on Jan. 29, 2024.

(51) Int. Cl. A61B 17/04; A61B 17/00; A61B 90/00

(52) U.S. Cl. CPC A61B 17/0401; A61B 90/03; A61B 2017/00955; A61B 2017/042

(57) ABSTRACT

Methods for fixating an implant having thermoplastic properties in a recess of an object, wherein the device comprises a stop member configured to stop a movement of the device when fixating the implant.

### CROSS-REFERENCE TO RELATED APPLICATIONS

This application claims the benefit of and priority to U.S. Provisional Application No. 63/626,292, filed Jan. 29, 2024, and is a continuation-in-part application of U.S. application Ser. No. 18/605,571, filed Mar. 14, 2024, both of which are incorporated by reference herein in their entirety for all purposes.

### FIELD

The current invention relates to a method for fixating an implant having thermoplastic properties in a recess of an object.

### BACKGROUND

Known embodiments of a sonotrode for fixating an implant with thermoplastic properties in a recess of an object, e.g. a bone, have implant size-specific shapes and sizes, which means that different sonotrodes are required for different implant sizes (see, e.g., WO 2012/037699). Known devices using ultrasound for fixating an implant in a recess of an object, e.g. a bone, share the drawback that the insertion depth in the recess is controlled manually-typically with visual control—while relying on the skills of the person performing the insertion (see, e.g., WO 2012/037699).

### SUMMARY

It is a task of current invention to provide an improved method for implantation of implants.

In an aspect, the present invention is directed to sonotrode for fixating an implant having thermoplastic properties in a recess of an object, wherein the sonotrode comprises a body having a proximal end for attaching an ultrasound generating device, a distal end, a tapered tip at the distal end of the body, wherein the tapered tip is tapered towards a distal end of the tip and wherein the tapered tip is configured to receive an implant having thermoplastic properties by inserting the tip at least partially into a cavity of the implant, and a z-axis from the distal end of the tapered tip to the proximal end of the body, wherein an area of the tapered tip at the distal end of the body projected onto a plane normal to the z-axis fully covers an area of the distal end of the body projected onto the plane normal to the z-axis.

The definitions and explanations given for the terms and features of the present invention apply to all aspects and embodiments disclosed herein, unless specifically indicated otherwise.

The sonotrode for use in the present invention refers to a connecting element between an ultrasound generator and an object to which the ultrasound is transmitted, which is, e.g., the implant in the present case. The sonotrode transmits vibrational energy, i.e. is suitable for the transmission of vibrational energy, from the ultrasound generator to an object, e.g. the implant. For example, the sonotrode has an elongated body from its proximal end to its distal end. It is noted that the ultrasound is at least partially transmitted to the implant; for example, not a full amplitude may be transmitted from the sonotrode to the implant but rather the ultrasonic impulse is transmitted to the implant. In any case, transmission is such that the implant having thermoplastic properties is at least partially liquified by the ultrasound transmitted from the sonotrode, e.g. to the extent that fixation of the implant in a recess of an object is achieved after solidification of the implant.

The ultrasound generating device referred to in here can be any known ultrasound generating device suitable for generating vibrational energy that can be transmitted by a sonotrode. In particular, the ultrasound generating device generates vibrational energy that, after transmission via the sonotrode, is suitable for liquifying a thermoplastic component, e.g. the implant as described herein, attached to the sonotrode.

The sonotrode of the present invention has a tapered tip that is configured to receive an implant having thermoplastic properties. The tapered tip is tapered from the distal end of the body to the distal end of the tip (i.e. tapered over the entire length of the tip from the distal end of the body to the distal end of the tip), in other words, it is tapered “away” from the body. The term “implant having thermoplastic properties” refers to any object (e.g. suture anchor or pin) that is suitable for implantation into another object, and having at least partial thermoplastic properties. In other words, the implant comprises a thermoplastic material which imparts thermoplastic properties such that at least a part of the implant can be liquified by the vibrational energy applied to the implant via the sonotrode, in particular during the insertion of the implant into the recess, where the implant will re-solidify due to cooling. Materials having thermoplastic properties suitable for the implant as described herein are known in the art and include, e.g., thermoplastic polymers, e.g.: resorbable or degradable polymers such as polymers based on lactic and/or glycolic acid (PL A, PLLA, PGA, PLGA etc.) or polyhydroxy alkanoates (PHA), polycaprolactone (PCL), polysaccharides, polydioxanes (PD) polyanhydrides, polypeptides or corresponding copolymers or composite materials (e.g. organic or inorganic fibers or whiskers, e.g. of calcium phosphate ceramics or glasses) containing the named polymers as a component; or non-resorbable or non-degradable polymers such as polyolefines (e.g. polyethylene), polyacrylates, polymetacrylates, polycarbonates, polyamides, polyester, polyurethanes, polysulfones, polyarylketones, polyimides, polyphenyl sulfides or liquid crystal polymers LCPs, polyacetales, halogenated polymers, in particular halogenated polyolefines, polyphenylensulfides, polysulfones, polyethers or equivalent copolymers or composite materials containing the named polymers as a component. For further exemplary thermoplasts, see, e.g., WO 2012/037699, which is incorporated by reference in its entirety, in particular page 6, line 8 to page 9, line 7.

The implant (e.g. suture anchor or pin) having thermoplastic properties can have any form or shape as long as it is suitable for being received by the sonotrode and for being inserted and fixated in a recess of an object. “Receive (d)”, as used herein, means that the implant can be physically held by the tip of the sonotrode and a transmission of vibrational energy from the sonotrode to the implant can take place to the extent that liquification of at least part of the thermoplastic components of the implant can occur by means of the vibrational energy transmitted. For example, the implant referred to in here has a cavity, recess, opening (which terms are used interchangeably) or elongated cavity, recess, opening or hole (e.g. an axially (along the z-axis) extended opening) which can interact with the tapered tip of the sonotrode so that the implant can be received by the sonotrode tip (see, e.g., FIG. 3). The implant can, e.g., be a suture anchor or a pin which can be fixated in an object. For example, the implant can be a suture anchor as described in WO 2012/037699, incorporated by reference in its entirety, in particular as described therein on page 2, lines 9 to 16, and pages 3, line 23 to page 4, line 23. For example, the implant as described herein for all aspects and embodiments can be a suture anchor or pin that has an overall cylindrical shape, for example the shape of a dowel. At the distal tip of this suture anchor or dowel, there may be a suture conduit or hole. The suture anchor or pin in this example features a cavity, recess, opening as described above into which the tapered tip can be inserted. For example, the suture anchor may be an essentially cylindrical structure with a length of 4 mm to 20 mm, an outer diameter of 1 mm to 9 mm, and with a cavity that stretches from the proximal end into the suture anchor towards the distal end over 0.5 mm to 8 mm and having a cavity diameter of 0.2 mm to 8 mm. The cavity may be cylindrical or polygonal (prismatic) and/or have a tapered (conus) shape (with the largest diameter at the proximal end of the suture anchor). The pin as described herein may have the same dimensions as the anchor or have the following dimensions: length of 4 mm to 60 mm, outer diameter of 1 mm to 10 mm, cavity depth (from the proximal end into the pin towards the distal end) of 0.5 mm to 10 mm, cavity diameter 0.2 mm to 8 mm.

For example, the implant (e.g. the suture anchor or pin) for all aspects and embodiments disclosed herein, features a cavity, recess or opening (in z-axis direction when received by the sonotrode (tip)) that is dimensioned such that a distal end of the sonotrode tip does not reach the end of the cavity (in z-axis direction) in the absence of ultrasound (see FIG. 4B). Hence, the cavity depth or length can be defined relative to the sonotrode tip- or vice-versa—in that the cavity is longer, e.g. about 5% to 50% longer, than the part of the sonotrode tip that inserts into the cavity in the absence of ultrasound generation. For example, the cavity is clear of the sonotrode tip (in the absence of ultrasound) for a distance of about 0.1 to 5 mm measured from the distal end of the sonotrode tip inserted into the cavity to the end of the cavity in y-axis direction before ultrasound is generated. This geometry can allow that upon ultrasound generation, the tip can insert a bit further into the implant's cavity, thus improving liquification of the implant.

For example, the implant (e.g. the suture anchor or pin) for all aspects and embodiments disclosed herein, features a cavity, recess or opening (in z-axis direction when received by the sonotrode (tip)) that is tapered from a proximal end of the implant towards a distal end (the wider part of the opening at the proximal end). The tapered cavity can have the shape of a tapered cylinder or it can be of a tapered polygonal or prismatic shape. For example, the tapered tip of the sonotrode and/or the tapered cavity of the implant (e.g. suture anchor or pin) are shaped complementary, i.e. the (negative) shape of the cavity corresponds to the (positive) shape of at least part of the sonotrode tip, and they may, for example, share the same taper angle. For example, the tapered tip of the sonotrode and/or the tapered cavity of the implant (e.g. suture anchor or pin) is dimensioned such that when the tapered tip is inserted into the tapered cavity, the tapered tip contacts at least 50% or at least 80% of the cavity surface in the implant. An exemplary implant with a tapered cavity may have the following dimensions: implant, optionally suture anchor, with an essentially cylindrical structure with a length of 4 to 20 mm, an outer diameter of 1 to 9 mm, and with a tapered cavity that stretches from the proximal end (widest point) into the suture anchor towards the distal end over 0.5 to 8 mm and having a largest diameter of 0.1 to 8 mm (proximal end), a smallest diameter of 0.05 to 3 mm and/or a taper angle of 1 to 20 degrees. The suture anchor may be dimensioned such that the sonotrode tip does not reach the end of the cavity when inserted, as described above. The pin as described herein may have a tapered cavity and may have have the following dimensions: a length of 4 mm to 60 mm, an outer diameter of 1 mm to 10 mm, a cavity depth (from the proximal end (widest point) into the pin towards the distal end) of 0.5 mm to 10 mm, a largest cavity diameter (proximal end) of 0.2 mm to 9 mm, a smallest cavity diameter of 0.1 mm to 4 mm, and/or a taper angle of 1 to 20 degrees. The pin may be dimensioned such that the sonotrode tip does not reach the end of the cavity when inserted, as described above.

The outer circumferential surface of the implant, e.g. suture anchor or pin, may be irregular and/or, e.g., feature protrusions, e.g. as detailed in WO 2012/037699. In an example, the proximal end of the implant (suture anchor or pin) for use in the present invention has an essentially flat surface, e.g. forming a flat surface perpendicular to the axis of tip insertion (along the z-axis), which axis can correspond to the direction of elongation of the implant (suture anchor or pin) and its cavity.

The object, as used herein, can, for example, be a human or animal hard tissue (e.g. bone), a cartilage-covered hard tissue (e.g. cartilage-covered bone), or an implant structure (e.g. fixation plate or bone plate) made, e.g. from titanium, steel, non-resorbable or resorbable polymer material or a fiber composite material. The hard tissue can, for example, be an artificial hard tissue, a hard tissue sample outside of the human or animal body, a hard tissue of a dead body or a hard tissue of a human or animal patient. It is noted that in particular implants having thermoplastic properties for being fixated in the context of surgery on humans or animals have a limited size range, which allows the skilled person to clearly assess the suitability of the present tapered tip to be configured to receive an implant having thermoplastic properties by inserting the tip at least partially into a cavity of the implant.

The recess in the object, into which the implant is fixated, can be an elongated recess, e.g. as obtained by drilling with a drill, for example a surgical drill.

An area of the tapered tip at the distal end of the body projected onto a plane normal to the z-axis fully covers an area of the distal end of the body projected onto the plane normal to the z-axis. In other words, the area of the tapered tip can be larger than the area of the distal end of the body, but not vice-versa. If viewed along the z-axis from the tapered tip towards the proximal end of the body, the body does not have a wider area, e.g. cross-section, than the largest area of the tapered tip where the tapered tip meets the body (which is where the tapering ends). The area refers to the total area of the tapered tip at the distal end of the body and to the total area of the distal end of the body, e.g. as illustrated in FIG. 1B.

As used herein, projected areas also include hollow sections in the profile, i.e. within the tip and/or body. The body may, for example, feature one or more sections which have an area projected onto the plane normal to the z-axis that is larger than the area of the tapered tip at the distal end of the body projected onto the plane normal to the z-axis, as long as these sections do not coincide with the distal end of the body (where the tapered tip is) but are, for example, located further in direction of the proximal end of the body, e.g. such that no physical contact between this one or more sections and an implant received by the tapered tip occurs (see, e.g. sonotrodes in FIGS. 2(b) and 3). It is further noted that the sonotrode may feature further structures that are added concentrically with the sonotrode body as long as these structures do not contact an implant when received by the sonotrode.

The vibrational energy is transferred to an implant when attached to the tip via the surface of the sonotrode tip that is in physical contact with the implant received by the sonotrode tip. Contrary to prior art sonotrodes, the sonotrode of the present invention-due to its shape—can physically and vibrationally interact with the implant (when received by the tip) exclusively via the surface of the tip that can receive the implant, i.e. is “inserted” into the implant. In other words, the sonotrode is configured such that it does not physically contact an area of the implant outside of where the tip receives the implant. For example, the implant has an axially extended opening which aligns with the z-axis of the sonotrode into which the sonotrode tip is inserted. The sonotrode, due to its shape, is configured such that it does not interact, e.g. touch, the circumference of the opening of the implant (or any other surface of the implant outside the opening) when received by the tip.

It was surprisingly found that the geometry of the sonotrode of the present invention is not only effective for transmitting a sufficient amount of vibrational energy for liquification of the thermoplastic components of the implant, but is also suitable for receiving implants of different sizes. Depending on the size, e.g., of the opening in the implant by which the sonotrode tip receives the implant, and/or of the entire implant, the same tapered tip of the sonotrode will insert at different depths of differently sized openings of differently sized implants. This is in contrast to prior art sonotrodes which feature a wider body area at the proximal end of the tapered tip which area contacts at least the circumference of the opening of the implant for transmitting vibrational energy which in turn means that a given sonotrode is size-specific for a given implant (see, e.g. FIG. 6A, FIG. 6B and FIG. 6C). Hence, with the present sonotrode, the operator (e.g. surgeon) does not need to change the sonotrode (or sonotrode tip) when handling implants of different sizes. A further advantage of the present sonotrode is that its geometry reduces or even avoids contact of the (vibrating) sonotrode, e.g. with sensitive tissue (e.g. cartilage) located around or on top of the location (e.g. a recess or opening in a hard tissue such as a bone) where the implant is fixated. Additionally, the geometry avoids the situation where a suture of a suture anchor would be caught between the surface of an object (e.g. bone) of insertion and the sonotrode, which could lead to a damage of the suture.

Also disclosed is a sonotrode for fixating an implant, optionally having thermoplastic properties, in a recess of an object, wherein the sonotrode comprises a body having
a proximal end for attaching an ultrasound generating device,
a distal end,
a tapered tip at the distal end of the body, wherein the tapered tip is tapered towards a distal end of the tip, and
a longitudinal z-axis from the distal end of the tapered tip to the proximal end of the body,
wherein an area of the tapered tip at the distal end of the body projected onto a plane normal to the z-axis fully covers an area of the distal end of the body projected onto the plane normal to the z-axis.

In an embodiment of the sonotrode according to the present invention, which may be combined with any of the embodiments of the sonotrode preaddressed or still to be addressed unless in contradiction, the area of the body projected onto the plane normal to the z-axis is constant along the z-axis from the proximal end of the body to the distal end of the body or decreases along the z-axis from the proximal end of the body to the distal end of the body.

As outlined above, when viewed along the z-axis from the tapered tip towards the proximal end of the body, the body does not have a wider area, e.g. cross-section, than the largest area of the tapered tip where the tapered tip meets the body. This geometry ensures that the sonotrode interacts with the implant exclusively via the surface of the tip that receives the implant, i.e. is “inserted” into the implant and does not interact, e.g. touch, the circumference of the opening of the implant. It is also within the scope of the present invention that the area of the body projected onto the plane normal to the z-axis is constant or decreases along the z-axis from the distal end of the body, i.e. where the tapered tip and the body are joined, to the proximal end of the body.

In an embodiment of the sonotrode according to the present invention, which may be combined with any of the embodiments of the sonotrode preaddressed or still to be addressed unless in contradiction, the body is essentially cylindrical and a largest diameter of the tapered tip is the same or larger than the diameter of the body at the distal end of the body.

In an embodiment of the sonotrode according to the present invention, which may be combined with any of the embodiments of the sonotrode preaddressed or still to be addressed unless in contradiction, the tapered tip has a cone shape and/or ends in a flat tip or in a pointy tip.

In an embodiment of the sonotrode according to the present invention, which may be combined with any of the embodiments of the sonotrode preaddressed or still to be addressed unless in contradiction, the body and the tapered tip are one piece or two pieces, wherein the two pieces are separable or not separable. The term “separable” as used herein means that the body and the tapered tip can be separated without destruction and optionally reversibly, i.e. the body and the tip can be detached and reattached, e.g. by a plug-fit connection, by clip-on connection, by a screw connection, by a bayonet fitting or similar connections. Alternatively, the body and tip can be “not separable”, i.e. the body and tip form a monobloc or are attached to each other by welding or gluing.

In an embodiment of the sonotrode according to the present invention, which may be combined with any of the embodiments of the sonotrode preaddressed or still to be addressed unless in contradiction, the body is prismatic or essentially cylindrical and has a diameter of 2 mm to 20 mm. As used herein, the diameter of the body is the largest extension of the body perpendicular to the axis of the body from the proximal to the distal end.

In an embodiment of the sonotrode according to the present invention, which may be combined with any of the embodiments of the sonotrode preaddressed or still to be addressed unless in contradiction, the tapered tip has a cone shape and at least one of: a largest diameter of 2 mm to 8 mm, a smallest diameter of 0.1 mm to 3 mm, a length along the z-axis of 1 mm to 10 mm, and a taper angle α of 1 degree to 20 degrees.

The above numbers refer to the tapered tip, i.e. the part of the sonotrode that includes a tapering. The taper angle α as used herein refers to the angle between the central axis and the tangential axis to the tapered surface (see FIG. 1C).

In an embodiment of the sonotrode according to the present invention, which may be combined with any of the embodiments of the sonotrode preaddressed or still to be addressed unless in contradiction, the body is essentially cylindrical, the tapered tip has a cone shape and the sonotrode has at least one of: a largest diameter of 2 mm to 20 mm, a smallest diameter of 0.1 mm to 3 mm, a length along the z-axis of 5 mm to 400 mm, and a taper angle α of the tapered tip of 1 degree to 20 degrees. Optionally, the length of the sonotrode along the z-axis can also correspond to the formula

$$l = n * ( \frac{λ}{2} ) ,$$

wherein 1 is the length of the sonotrode, n is an integer from 1 to 6 and A is the resonance length of the sonotrode at the ultrasound frequency generated by the ultrasound generating device in kHz, optionally at a frequency from 15 kHz to 45 kHz, optionally 20 kHz to 30 KHz.

The above numbers refer to the tapered tip, i.e. the part of the sonotrode that includes a tapering and the taper angle x is as define above.

In an embodiment of the sonotrode according to the present invention, which may be combined with any of the embodiments of the sonotrode preaddressed or still to be addressed unless in contradiction, the sonotrode is suitable to operate between 15 kHz and 45 kHz.

In an embodiment of the sonotrode according to the present invention, which may be combined with any of the embodiments of the sonotrode preaddressed or still to be addressed unless in contradiction, the sonotrode is configured to have an amplitude at the tapered tip of 10 μm to 45 μm when operated between 10 and 45 kHz and 5 to 150 W.

The amplitude refers to an amplitude in direction of the z-axis. In an embodiment of the sonotrode according to the present invention, which may be combined with any of the embodiments of the sonotrode preaddressed or still to be addressed unless in contradiction, the sonotrode is made of steel, titanium, cobalt-based alloys, ceramic, silicon nitride, silicon carbide, zirconia, or an aluminum-zirconia alloy.

In an embodiment of the sonotrode according to the present invention, which may be combined with any of the embodiments of the sonotrode preaddressed or still to be addressed unless in contradiction, the sonotrode further comprises an implant having thermoplastic properties plugged onto the tapered tip, wherein the sonotrode and the implant are configured such that the tapered tip is exclusively in contact with a cavity in the implant.

The implant and its interaction with the tip was described above. It is again noted that the sonotrode is attached to, i.e. plugged into, the implant such that (a) the implant is physically held in place at the tip of the sonotrode at least during the handling of the implant in handling or processing steps leading to the fixation of the implant in a recess of an object, and (b) the ultrasound is at least partially transmitted to the implant; for example, not a full amplitude may be transmitted from the sonotrode to the implant but rather the ultrasonic impulse is transmitted to the implant. In any case, transmission is such that the implant having thermoplastic properties is at least partially liquified by the ultrasound transmitted from the sonotrode, e.g. to the extent that fixation of the implant in a recess of an object is achieved after solidification of the implant. The cavity in the implant is as defined above, i.e. it is a cavity, recess, opening or elongated cavity, recess, opening or hole (e.g. an axially (direction z-axis) extended opening) extending from a surface of the implant into a body of the implant, which cavity can interact with the tapered tip of the sonotrode so that the implant can be received by the sonotrode tip.

In an embodiment of the sonotrode according to the present invention, which may be combined with any of the embodiments of the sonotrode preaddressed or still to be addressed unless in contradiction, the cavity in the implant is an axially extended opening in direction of the z-axis.

In an embodiment of the sonotrode according to the present invention, which may be combined with any of the embodiments of the sonotrode preaddressed or still to be addressed unless in contradiction, the tapered tip and the implant are configured such that implants of different sizes can be plugged onto the tapered tip.

As explained above, the tapered tip of the sonotrode is suitable for receiving implants of different sizes. Depending on the size, e.g., of the opening (cavity) in the implant by which the sonotrode tip receives the implant, and/or of the entire implant, the same tapered tip of the sonotrode will insert at different depths in differently sized openings of differently sized implants, which allows for differently sized implants to be received by and plugged onto the tapered tip.

In an embodiment of the sonotrode according to the present invention, which may be combined with any of the embodiments of the sonotrode preaddressed or still to be addressed unless in contradiction, the implant having thermoplastic properties is a pin or a suture anchor.

In a further aspect, the present invention is directed to an apparatus for fixating an implant in a recess of an object comprising the sonotrode as described herein and an ultrasound generating device attached to the proximal end of the body.

The definitions provided above in the context of the sonotrode apply to the aspect of the apparatus. In particular, the ultrasound generating device (as defined above) is configured to generate vibrational energy and is attached to the sonotrode such that the vibrational energy can be transmitted via the attached sonotrode. The vibrational energy is generated and transmitted such that a thermoplastic component, e.g. the implant (e.g. a suture anchor or pin) as described herein, which can be attached to the sonotrode can be liquified (as described above).

In an embodiment of the apparatus according to the present invention, which may be combined with any of the embodiments of the apparatus preaddressed or still to be addressed unless in contradiction, the apparatus is configured to at least one of: operate between 10 kHz and 45 kHz, have an amplitude at the tapered tip of 10 μm to 45 μm when operated between 10 kHz and 45 kHz and 2 W to 150 W, or both.

In an embodiment of the apparatus according to the present invention, which may be combined with any of the embodiments of the apparatus preaddressed or still to be addressed unless in contradiction, the apparatus further comprises an implant having thermoplastic properties, optionally a pin or a suture anchor, plugged onto the tapered tip, optionally wherein the tapered tip is exclusively in contact with an axially extended opening in the implant, optionally pin or anchor, in direction of the z-axis.

As explained above, the implant (e.g. pin or suture anchor) has an axially extended opening which aligns with the z-axis of the sonotrode into which the sonotrode tip is inserted. The sonotrode, due to its shape, is configured such that it does not interact, e.g. touch, the circumference of the opening of the implant (or any other surface of the implant outside the opening) when received by the tip. The interaction between the sonotrode tip and the implant (e.g. pin or suture anchor) is effective for transmitting a sufficient amount of vibrational energy for liquification of the thermoplastic components of the implant, and the tip is suitable for receiving implants of different sizes.

In an embodiment of the apparatus according to the present invention, which may be combined with any of the embodiments of the apparatus preaddressed or still to be addressed unless in contradiction, the implant is a suture anchor that has a suture conduit at a distal end of the anchor. As described, suture anchors are known in the art and the suture anchor may, optionally, comprise a suture in the suture conduit when attached to the apparatus.

In a further aspect, the present invention is directed to a kit of parts comprising the sonotrode described herein and an ultrasound generating device suitable for attachment to the proximal end of the body. Essentially, the kit of parts comprises some or all of the individual components described for the apparatus and all definitions provided herein for the aspect of the sonotrode and the apparatus apply to the kit of parts. The kit of parts may optionally further comprise instructions for assembly of the parts and/or operation of the assembled parts.

In an embodiment of the kit of parts according to the present invention, which may be combined with any of the embodiments of the kit of parts preaddressed or still to be addressed unless in contradiction, the kit of parts further comprises at least one implant having thermoplastic properties, optionally a pin or suture anchor, suitable for being plugged onto the tapered tip. In an embodiment of the kit of parts according to the present invention, which may be combined with any of the embodiments of the kit of parts preaddressed or still to be addressed unless in contradiction, the kit comprises at least two implants, optionally pins or suture anchors, of different sizes. As explained above, the sonotrode of the kit of parts is not only effective for transmitting a sufficient amount of vibrational energy for liquification of the thermoplastic components of the implant, but is also suitable for receiving implants of different sizes. Depending on the size, e.g., of the opening in the implant by which the sonotrode tip receives the implant, and/or of the entire implant, the same tapered tip of the sonotrode will insert at different depths in differently sized openings of differently sized implants. Hence, the operator (e.g. surgeon) does not need to change the sonotrode (or sonotrode tip) when handling implants of different sizes.

In an embodiment of the kit of parts according to the present invention, which may be combined with any of the embodiments of the kit of parts preaddressed or still to be addressed unless in contradiction, the at least one implant or the at least two implants is/are a pin or suture anchor, wherein the tapered tip and the pin or suture anchor are configured such that the tapered tip is exclusively in contact with an axially extended opening in the pin or anchor in direction of the z-axis.

In an embodiment of the kit of parts according to the present invention, which may be combined with any of the embodiments of the kit of parts preaddressed or still to be addressed unless in contradiction, the suture anchor has a suture conduit at a distal end of the anchor.

In an embodiment of the kit of parts according to the present invention, which may be combined with any of the embodiments of the kit of parts preaddressed or still to be addressed unless in contradiction, the suture anchor has a suture conduit at a distal end of the anchor, and wherein the kit of parts further comprises a suture suitable for being threaded through the suture conduit or being threaded through the suture conduit.

In a further aspect, the present invention is directed to a method for assembling a device for fixating at least one implant having thermoplastic properties in a recess of an object, optionally a human or animal hard tissue, a cartilage-covered hard tissue, or an implant structure (e.g. a fixation plate or bone plate), the method comprising the steps of:
providing the sonotrode as described herein, an ultrasound generating device and at least one implant having thermoplastic properties, optionally a pin or suture anchor, and in any order:
attaching the ultrasound generating device to the proximal end of the body, and
plugging a proximal end of the at least one implant onto the tapered tip of the sonotrode, in particular wherein the sonotrode and the implant are configured such that the tapered tip is exclusively in contact with an axially extended opening in the implant in direction of the z-axis.

The definitions provided above in the context of the sonotrode, the apparatus and the kit of parts apply to the aspect of the method for assembling a device for fixating at least one implant having thermoplastic properties in a recess of an object. Typically, the ultrasound generating device is attached to the proximal end of the body before the implant is plugged onto the tapered tip. However, the inverse order is also encompassed by the present method.

In an embodiment of the method for assembling a device for fixating at least one implant having thermoplastic properties according to the present invention, which may be combined with any of the embodiments of the method preaddressed or still to be addressed unless in contradiction, the implant is a pin or a suture anchor, optionally a suture anchor that comprises a suture threaded through a suture conduit at a distal end of the suture anchor, or wherein the method further comprises threading a suture through a suture conduit at a distal end of the suture anchor.

In a further aspect, the present invention is directed to a method for fixating at least one implant, optionally a pin or suture anchor, optionally at least one suture with at least one suture anchor, in a recess of an object, the method comprising the steps of:
(a) providing the apparatus as described herein, or providing the sonotrode as described herein and attaching an ultrasound generating device to the proximal end of the body,
(b) providing at least one implant having thermoplastic properties, optionally at least one pin having thermoplastic properties or at least one suture anchor having thermoplastic properties and comprising a suture in a conduit at a distal end of the anchor or providing at least one suture anchor having thermoplastic properties and threading a suture through a conduit at a distal end of the suture anchor,
(c) plugging the at least one implant, optionally the at least one pin or suture anchor, onto the tapered tip of the sonotrode, in particular such that the tapered tip is exclusively in contact with an axially extended opening in the implant, optionally pin or anchor, in direction of the z-axis, and
(d) providing an object with a recess, wherein the recess: has a cross-section corresponding to the cross-section of the at least one implant, optionally pin or suture anchor, or
has a cross-section that is smaller than the cross-section of the at least one implant, optionally pin or suture anchor, to the extent that an insertion of the at least one implant, optionally pin or suture anchor, is possible, or
has a cross-section corresponding to or being larger than the cross-section of the implant, optionally pin or suture anchor, and has a depth that is shorter than the length of the implant, optionally pin or suture anchor, in z-direction, followed by
(e) inserting the at least one implant, optionally pin or suture anchor, into the recess of the object while or followed by transmitting ultrasound from the sonotrode to the at least one implant, optionally pin or suture anchor, for a time sufficient to liquify the at least one implant, optionally pin or suture anchor, to the extent that liquification results in fixation of the implant, optionally pin or suture by means of the at least one anchor, in the recess.

The definitions provided above in the context of the sonotrode, the apparatus, the kit of parts and the method for assembling a device for fixating at least one implant having thermoplastic properties in a recess of an object apply to the aspect of the method for fixating at least one implant, optionally pin or suture anchor, optionally suture with at least one suture anchor, in a recess of an object. Steps (b), (c) and (d) can be performed in this order or in any other order, e.g. (d), (b), (c) or any permutation thereof. Steps (c) and (e) may be repeated on the same or different object with the same or differently sized implants, optionally pins or suture anchors, e.g. while using the same sonotrode.

The recess in the object as described in step (d) may be obtained by drilling into the object with a drill of a suitable diameter (i.e. cross-section), e.g. a diameter corresponding to the cross-section of the at least one implant, optionally pin or suture anchor, or having a cross-section that is larger or smaller than the cross-section of the at least one implant, optionally pin or suture anchor. Additionally, and in particular, the recess can be drilled such or the implant can be chosen such relative to the drilled recess that the depth of the recess (how far the recess is drilled into the object) is shorter than the length of the implant. The length of the implant refers to the distance between end points of the implant in direction of insertion into the recess, which typically corresponds to the direction along the axis of attachment of the implant to the sonotrode (tip). It is within the purview of the skilled person to choose and install suitable recess cross-sections which will allow for implant insertion and fixation by means of liquification and re-solidification.

In step (e), the implant, optionally pin or suture anchor according to the invention is forced into the recess of the object (e.g. a hard tissue opening) and the liquification energy is transmitted via the sonotrode either simultaneously with implant insertion into the recess or after the implant is positioned in the recess, the latter is particularly preferred if the recess has a cross-section corresponding to or being larger than the cross-section of the implant and the recess has a depth that is shorter than the length of the implant. The term “to liquify the at least one implant, optionally pin or suture anchor” means that the implant, optionally pin or suture anchor is at least partially liquified to the extent that the thermoplastic components/materials of the implant liquify in order to allow for the fixation of the implant in the recess.

Generally, and in an example, the liquified implant extends or can flow into the bone structure surrounding the recess (e.g. because of the porous nature of the bone structure) when implanted and liquified by ultrasound. For example, the difference in cross-section between the implant and the recess into which the implant is implanted can influence where the implant, once liquified, extends into the bone structure. For example, if the implant cross-section and the recess cross-section are essentially the same, or if the recess cross-section is slightly smaller than the implant cross-section, a part of the liquified implant can “flow into”, i.e. extend into, the bone structure at the wall of the recess and optionally also at the bottom of the recess. If the cross-section of the implant is smaller than the cross-section of the recess, the liquified implant will rather or to a greater extent extend into the bone at the bottom of the recess and to a lesser extent or not at all at the wall of the recess. Therefore, it is within the purview of the present invention that the selection of implant and recess cross-sections can be used in the present method to control the location where the liquified implant extends into the bone, at least to a relative extent, i.e. whether the liquified implant extends more into the bone at the wall of the recess or at the bottom of the recess. The selection of the location in the recess where the implant predominantly extends into the bone structure can be of advantage, e.g., if the bone structure differs between the wall of the recess and the bottom of the recess. For example, the flow of the implant into the bone structure can be controlled by the relative cross-sections of the recess and the implant such that it flows into bone areas of greater stability which will improve fixation of the implant or, e.g., of a suture with the implant.

In an embodiment of the method for fixating at least one implant, optionally pin or suture anchor, optionally at least one suture with at least one suture anchor, in a recess of an object according to the present invention, which may be combined with any of the embodiments of the method preaddressed or still to be addressed unless in contradiction, after step (e) the method comprises repeating step (b) with a further implant, optionally pin or suture anchor of the same or of a different size compared to the at least one implant, optionally pin or suture anchor, of previous step (b), followed by repeating steps (c) to (e) with the further implant, optionally pin or suture anchor, wherein the steps (b) and (c) to (e) are repeated at least once in this sequence.

As outlined above and for example, the same sonotrode or apparatus of step (a)—i.e. the same sonotrode—can be used for the repetition of step (b), followed by the repetition of steps (c) to (e), with differently sized implants (e.g. pins or suture anchors). Hence, the operator (e.g. surgeon) does not need to change the sonotrode (or apparatus) when handling implants of different sizes during the repetition of step the method steps. Alternatively, a differently sized sonotrode or apparatus can be provided before repeating step (b).

In an embodiment of the method for fixating at least one implant, optionally pin or suture anchor, optionally at least one suture with at least one suture anchor, in a recess of an object according to the present invention, which may be combined with any of the embodiments of the method preaddressed or still to be addressed unless in contradiction, the object is a hard tissue, a cartilage-covered hard tissue, or an implant structure.

In an embodiment of the method for fixating at least one implant, optionally pin or suture anchor, optionally at least one suture with at least one suture anchor, in a recess of an object according to the present invention, which may be combined with any of the embodiments of the method preaddressed or still to be addressed unless in contradiction, the hard tissue is an artificial hard tissue, a hard tissue sample outside of the human or animal body, a hard tissue of a dead body or a hard tissue of a human or animal patient, and/or the implant structure is a fixation plate or bone plate.

The fixation plate or bone plate can be fixated to a hard tissue, e.g. a bone. For example, the fixation plate or bone plate can be made of titanium, steel, non-resorbable or resorbable polymer material or a fiber composite material, and is, e.g., used to fixate fractures or osteotomies. A hole can be inserted into the plate to allow for insertion of a pin (or suture anchor) as described herein, which, if the plate is made of polymer material can also be welded together with the polymeric plate.

It is a further objective to provide a device for fixating an implant with thermoplastic properties in a recess of an object at a desired depth in the recess. In particular, it is an objective to fixate an implant having thermoplastic properties in a recess of an object with precise control of implantation depth and/or to fixate the implant at a predefined depth in the object.

This task is solved by methods with the features of claims 1, 2, 5 and 6. Further embodiments of the methods are defined by the features of further claims.

In an aspect, the present invention is directed to a device for fixating an implant having thermoplastic properties in a recess of an object, in particular at a predefined depth in the object, the device comprising:
an ultrasound generating device,
a sonotrode attached to a distal end of the ultrasound generating device at a proximal end of the sonotrode,
a stop member attached to or being part of the ultrasound generating device and being isolated against ultrasound, wherein
the sonotrode has a distal end configured to receive an implant having thermoplastic properties and has a y-axis extending from the proximal end to the distal end of the sonotrode,
the stop member is shorter than the sonotrode in y-axis direction from the proximal to the distal end of the sonotrode,
the stop member is configured to stop a movement of the device in y-axis direction by generating a mechanical resistance against a surface that is essentially perpendicular to the y-axis.

When fixating an implant in a recess of an object, e.g. a bone, it is advantageous to control how deep the implant is inserted and fixated in the recces in the object. Typically, the insertion depth in the recess is controlled manually-typically with visual control—by the person performing the insertion.

However, the fixation strength of the implant can depend on the insertion depth and manual control can be biased by the skill of the person performing the insertion. The device of the present invention allows for the precise control of insertion depth, which can be preset by adjusting the stop member, e.g. depending on the object into which the implant is inserted and/or the size of the implant. In the simplest example, the distance between the stop member and the sonotrode's distal end correlates with the insertion depth of the implant in the recess of an object (i.e. the depth in the object relative to the surface around the opening or cavity).

The definitions provided above in the context of the sonotrode, the apparatus, the kit of parts, the method for assembling a device for fixating at least one implant having thermoplastic properties in a recess of an object and the method for fixating at least one pin or at least one suture with at least one suture anchor in a recess of an object apply to the aspect of the device for fixating an implant having thermoplastic properties in a recess of an object, unless specifically stated otherwise.

The implant referred to is the implant characterized above in the context of the sonotrode, it is, e.g., a suture anchor or pin as explained above. Also, the details provided above for the ultrasound generating device apply to the ultrasound generating device of the device for fixating an implant. The ultrasound generating device may, e.g., be or form part of the handle of the device and may be attached to or form the stop member. The ultrasound generating device is functionally coupled to the sonotrode and may be (reversibly) detachable. The sonotrode can be any suitable element that transmits vibrational energy, i.e. is suitable for the transmission of vibrational energy, from the ultrasound generator to an object, e.g. the implant. The sonotrode's shape is not limited beyond functional restrictions and it can be any known sonotrode or, for example but not limited to, be a sonotrode as described (e.g., see above) or claimed herein. The sonotrode has a distal end suitable for receiving an implant having thermoplastic properties, for example by means of a tapered tip (for example a tapered tip as described herein in the context of the sonotrode). The distal end of the sonotrode (e.g. a tapered tip) can, for example, be at least partially inserted into a cavity of the implant, thereby plugging the implant onto the distal end (e.g. tip) of the sonotrode as discussed above. It is noted that the sonotrode can be a sonotrode as described (e.g., see above) or claimed herein. In this embodiment, the sonotrode's proximal end corresponds to the proximal end of the sonotrode body and the distal end of the sonotrode corresponds to the distal end of the body, wherein this distal end is further characterized in that it features a tapered tip. For example, the sonotrode of the device corresponds to the sonotrode body of the sonotrode as claimed herein, wherein the sonotrode as claimed herein is further characterized by the tapered tip and the z-axis. Regarding the z- and y-axes, it is noted that when the sonotrode as described (e.g., see above) or claimed herein is used in the device according to the present invention, these axes are parallel to each other and are superimposed, wherein one (y-axis) extends from the proximal to the distal end and the other one (z-axis) extends from the distal to the proximal end of the sonotrode (wherein the sonotrode corresponds to the sonotrode body).

The stop member is isolated against the ultrasound generated by the ultrasound generating device and against the ultrasound (vibrational energy) transmitted by the sonotrode. For example, isolation is achieved by attaching the stop member to the part of the ultrasound generating device that does not transmit vibrational energy and/or by a clearance (e.g. gap or space) between the stop member and the sonotrode. In other words, the stop member and the sonotrode are not in a physical contact that would allow for transmission of vibrational energy from the sonotrode to the stop member. For example, the stop member is also not indirectly in contact with the sonotrode via an implant attached to the sonotrode (tip). Additionally or alternatively, there may be an insulating material between the components of the device, in particular the sonotrode, and the stop member that insulates the stop member against vibrational energy, in particular from the sonotrode or the ultrasound generating device.

In order to allow for the sonotrode's distal end to reach the recess in the object, the stop member is shorter than the sonotrode in direction of the y-axis. Shorter means that if the stop member extends in direction of the y-axis, it does not reach the distal end of the sonotrode. In other words, at least part of the distal end of the sonotrode is clear of the stop member.

When implanting and fixating the implant in the recess of the object, the stop member is designed to stop the movement of the device in implantation direction, i.e. in y-axis direction, for example when reaching the surface around the recess of the object. The stop member is configured to generate a mechanical resistance against this surface when being pushed against the surface. For example, if the stop member is elongated along the y-axis, the mechanical resistance is brought about by the distal end of the stop member. The stop member is made of a material that is dimensionally stable to the extent that it can withstand manual pressure of a person operating the device against the surface without being significantly deformed (for example without changing the distance that is set by the stop member). Suitable materials include metals, plastics and composite materials such as glass fiber or carbon fiber materials. Exemplary materials include PEEK, polyamid, polycarbonate, polyester, glass fiber filled polyamid or polyester, and stainless steel. The term “essentially perpendicular to the y-axis” refers to any surface around the recess that is at an angle of about 90° to the recess axis or y-axis, e.g. between the angles of about 15° to 165°.

It is noted that the device and in particular the stop member is configured such that the stop member does not have physical contact with an implant received by the distal end of the sonotrode.

The object, as detailed above, can, for example, be a human or animal hard tissue (e.g. bone), a cartilage-covered hard tissue (e.g. cartilage-covered bone), or an implant structure (e.g. fixation plate or bone plate) made, e.g. from metal. Because the stop member is isolated against vibrational energy, the contact between the stop member and the surface (e.g. a sensitive surface such as cartilage, e.g. a 0.5-4.5 mm thick cartilage layer, on a hard tissue such as bone) does not significantly damage the surface. If, for example, the sonotrode as described (e.g., see above) or claimed herein is used for the device, this sonotrode, due to its shape, additionally avoids the contact between the sonotrode and any sensitive surface around the recess into which the implant is fixated.

For better understanding it is noted that the device is configured such that an implant received by the distal end of the sonotrode is inserted into and fixated in the recess of the object by transfer of a force from, e.g. exclusively, the sonotrode, and not from any other components of the device, in particular not from the stop member which has no physical contact with the implant.

In an embodiment of the device for fixating an implant having thermoplastic properties in a recess of an object according to the present invention, which may be combined with any of the embodiments of the device preaddressed or still to be addressed unless in contradiction, the stop member extends in direction of the distal end of the sonotrode along the y-axis. In other words, the stop member of this embodiment extends from the ultrasound generating device (for example where it is attached to the device) in direction of the distal end of the sonotrode, i.e. it extends along the sonotrode.

In an embodiment of the device for fixating an implant having thermoplastic properties in a recess of an object according to the present invention, which may be combined with any of the embodiments of the device preaddressed or still to be addressed unless in contradiction, the stop member at least partially or fully encloses the sonotrode around the y-axis. For example, the stop member may be a cylinder or a (e.g. round or polygonal) tube with the sonotrode within the cylinder or tube. Covering around the y-axis does not mean that the entire length of the sonotrode in y-axis direction is covered. “Partially encloses” means that the stop member may comprise a slot or other openings.

In an embodiment of the device for fixating an implant having thermoplastic properties in a recess of an object according to the present invention, which may be combined with any of the embodiments of the device preaddressed or still to be addressed unless in contradiction, the stop member is attached to the distal end of the ultrasound generating device. By being detachable, different stop members can be used for the same device, e.g. depending on implant size and/or desired depth of implantation/fixation in the recess in the object, and/or depending on whether a sleeve is used (see below). The stop member can be attached by any suitable mechanism that stably but removably attaches the stop member to the device, e.g. by a plug-fit connection, a clip-on connection, a screw connection or a bayonet mechanism.

In an embodiment of the device for fixating an implant having thermoplastic properties in a recess of an object according to the present invention, which may be combined with any of the embodiments of the device preaddressed or still to be addressed unless in contradiction, a proximal end of the stop member is attached to the distal end of the ultrasound generating device- or the stop member may be a part of the ultrasound generating device—the stop member has a distal end, the stop member extends in direction of the distal end of the sonotrode along the y-axis, and wherein a distance between the distal end of the sonotrode and the distal end of the stop member along the y-axis is in the range of 1 mm to 10 mm (for example if the stop member is contacting to bone directly). In an example where the device should be used on a cartilage surface, an exemplary distance is extended by the thickness of the cartilage, e.g. between 1 and 5 mm. If the device is used together with a sleeve in minimally invasive surgery, the distance is, e.g., extended by the length of the sleeve, for example from 25 mm to 40 mm (e.g. for applications in the hand/wrist and foot/ankle) to 100 mm to 300 mm, e.g. for arthroscopic applications in the shoulder or knee or on the spinal column.

In an embodiment of the device for fixating an implant having thermoplastic properties in a recess of an object according to the present invention, which may be combined with any of the embodiments of the device preaddressed or still to be addressed unless in contradiction, the stop member forms a or forms part of a handpiece of the device. For example, the stop member and optionally the ultrasound generating device may form or be part of the handpiece, i.e. the part of the device that is held by the person operating the device.

In an embodiment of the device for fixating an implant having thermoplastic properties in a recess of an object according to the present invention, which may be combined with any of the embodiments of the device preaddressed or still to be addressed unless in contradiction, the stop member forms a or forms part of a handpiece together with the ultrasound generating device.

In an embodiment of the device for fixating an implant having thermoplastic properties in a recess of an object according to the present invention, which may be combined with any of the embodiments of the device preaddressed or still to be addressed unless in contradiction, the stop member is separably attached to the ultrasound generating device.

In an embodiment of the device for fixating an implant having thermoplastic properties in a recess of an object according to the present invention, which may be combined with any of the embodiments of the device preaddressed or still to be addressed unless in contradiction, the device is configured to insert a pin or a suture anchor having thermoplastic properties in a recess of an object such that the pin or anchor sits flush with a surface around the recess of the object or such that there is a distance between a surface around the recess of the object and a proximal end of the pin or anchor of about 0.25 to 1.5 mm, optionally about 0.5 mm to 1 mm. The device can be configured accordingly by adjusting the stop member, in particular the stop member's extension in y-axis direction. The shorter the stop member is, the further the pin or suture anchor is inserted into the recess (greater implant depth).

In an embodiment of the device for fixating an implant having thermoplastic properties in a recess of an object according to the present invention, which may be combined with any of the embodiments of the device preaddressed or still to be addressed unless in contradiction, the device further comprises a sleeve slidably positioned around the sonotrode along the y-axis, wherein the sleeve has a distal and a proximal end, and is configured to stop a movement of the device in y-axis direction by generating a mechanical resistance at its distal end against a surface that is essentially perpendicular to the y-axis and at its proximal end against the stop member.

For example, the sleeve can be positioned with its distal end against the surface around the recess of the object (the term “perpendicular” is to be understood as explained in the context of the stop member). The sleeve is dimensionally stable, at least in y-axis direction, and can be made of a material as explained for the stop member. The sleeve may also be flexible or bendable to the extent that it is stable enough to set a distance between the surface around the recess in the object and the stop member. The device is configured such that when the device is moved in y-axis direction, the stop member stops the movement of the device by a mechanical resistance between the stop member and the proximal end of the sleeve. In other words, the proximal end of the sleeve (e.g. surface or rim thereof) is configured to generate a mechanical resistance or stop with the stop member (e.g. a surface or rim thereof). The sleeve is also isolated from vibrational energy as explained for the stop member. Either the sleeve's dimension (e.g. length along the y-axis), the stop member's dimension (e.g. length along the y-axis) or both can be adapted to adjust the implant depth, e.g. also depending on the implant size. The sleeve can have a cylindrical or prismatic (e.g. polygonal) shape and is dimensioned such that it can be positioned around the sonotrode, in particular such that it has no physical contact with the sonotrode to avoid the transmission of vibrational energy from the sonotrode to the sleeve. The sleeve can be used, e.g. in minimally invasive surgery or in open surgery.

In an embodiment of the device for fixating an implant having thermoplastic properties in a recess of an object according to the present invention, which may be combined with any of the embodiments of the device preaddressed or still to be addressed unless in contradiction, the sleeve has a slot from its distal end to its proximal end. The slot is, for example, dimensioned such that a suture from a suture anchor can be passed through the slot. For example, guiding the suture through the slot avoids that the suture is positioned between the proximal end of the sleeve and the stop member, or between the distal end of the sleeve and the surface, such that the suture could affect the distance between the proximal end of the sleeve and the stop member when the proximal end meets the stop member, or between the surface and the sleeve. This avoids an impact of the suture, and/or of differently sized (different thicknesses) sutures on the implant depth. The slot can extend along the entire sleeve (in y-axis direction) or it can be a partial slot at the distal end of the sleeve, as long as it is suitable for guiding the suture through the sleeve.

In an embodiment of the device for fixating an implant having thermoplastic properties in a recess of an object according to the present invention, which may be combined with any of the embodiments of the device preaddressed or still to be addressed unless in contradiction, the sleeve has at least one recess at its proximal end and/or at its distal end. The at least one recess may have any shape that is suitable for passing through a suture, e.g. of a suture anchor. This also avoids an impact of the suture, and/or of differently sized (different thicknesses) sutures on the implantation depth, as explained above.

Alternatively or additionally, the stop member 206 can comprise one or more recesses where the stop member meets the sleeve and/or the surface against which a mechanical resistance is generated. Thereby, it could also be achieved that the distance between the sleeve or surface and the stop member is not affected by the suture.

In an embodiment of the device for fixating an implant having thermoplastic properties in a recess of an object according to the present invention, which may be combined with any of the embodiments of the device preaddressed or still to be addressed unless in contradiction, the sonotrode is the sonotrode for fixating an implant having thermoplastic properties in a recess of an object as described herein.

The above embodiment is directed to the device described herein, in which the sonotrode for fixating an implant having thermoplastic properties in a recess of an object as described herein is used. In other words, in this embodiment, the device is one, wherein the sonotrode is a sonotrode comprising a body having
a proximal attached to the ultrasound generating device, which proximal end corresponds to the proximal end of the sonotrode defined in the context of the device,
a distal end, which distal end corresponds to the distal end of the sonotrode defined in the context of the device configured to receive an implant (with the additional limitation that the distal end has a tapered tip, as stated below)
a tapered tip at the distal end of the body-which means that the distal end of the sonotrode as defined in the context of the device has the form of a tapered tip—wherein the tapered tip is tapered towards a distal end of the tip and wherein the tapered tip is configured to receive an implant having thermoplastic properties by inserting the tip at least partially into a cavity of the implant, and
a z-axis from the distal end of the tapered tip to the proximal end of the body (the z- and y-axes are parallel to each other and are superimposed when the sonotrode as described or claimed herein is used in the device, wherein one (y-axis) extends from the proximal to the distal end and the other one (z-axis) extends from the distal to the proximal end of the sonotrode (wherein the sonotrode corresponds to the sonotrode body)), wherein an area of the tapered tip at the distal end of the body projected onto a plane normal to the z-axis fully covers an area of the distal end of the body projected onto the plane normal to the z-axis.

Hence, in the above embodiment, in which the device for fixating an implant having thermoplastic properties in a recess of an object is one, wherein the sonotrode is a sonotrode as described or claimed herein, the device comprises:
an ultrasound generating device,
a sonotrode having a body, wherein the sonotrode body is attached to a distal end of the ultrasound generating device at a proximal end of the sonotrode body,
a stop member attached to or being part of the ultrasound generating device and being isolated against ultrasound,
wherein
the sonotrode body has a distal end and a tapered tip at the distal end of the body, wherein the tapered tip is tapered towards a distal end of the tip and wherein the tapered tip is configured to receive an implant having thermoplastic properties by inserting the tip at least partially into a cavity of the implant, wherein the sonotrode body has a y-axis extending from the proximal end to the distal end of the sonotrode body, wherein the sonotrode body has a z-axis from the distal end of the tapered tip to the proximal end of the body, and wherein an area of the tapered tip at the distal end of the body projected onto a plane normal to the z-axis fully covers an area of the distal end of the body projected onto the plane normal to the z-axis,
the stop member is shorter than the sonotrode body in y-axis direction from the proximal to the distal end of the sonotrode body,
the stop member is configured to stop a movement of the device in y-axis direction by generating a mechanical resistance against a surface that is essentially perpendicular to the y-axis.

In a further aspect, the present invention is directed to a kit of parts comprising:
an ultrasound generating device,
a sonotrode attachable to a distal end of the ultrasound generating device at a proximal end of the sonotrode,
at least one stop member attachable to the ultrasound generating device and being isolated against ultrasound when attached to the ultrasound generating device, and
at least one implant having thermoplastic properties,
wherein
the sonotrode has a distal end configured to receive the at least one implant and has a y-axis extending from the proximal end to the distal end of the sonotrode,
the at least one stop member is shorter than the sonotrode in y-axis direction from the proximal to the distal end of the sonotrode,
the at least one stop member is configured to stop a movement of the device in y-axis direction by generating a mechanical resistance against a surface that is essentially perpendicular to the y-axis.

The definitions provided above in the context of the sonotrode, the apparatus, the kit of parts, the method for assembling a device for fixating at least one implant having thermoplastic properties in a recess of an object, the method for fixating at least one pin or at least one suture with at least one suture anchor in a recess of an object and the device for fixating an implant having thermoplastic properties in a recess of an object apply to the aspect of the kit of parts, unless specifically stated otherwise.

The kit of parts may optionally further comprise instructions for assembly of the parts and/or operation of the assembled parts.

In an embodiment of the kit of parts according to the present invention, which may be combined with any of the embodiments of the kit of parts preaddressed or still to be addressed unless in contradiction, the at least one implant is a pin or a suture anchor.

In an embodiment of the kit of parts according to the present invention, which may be combined with any of the embodiments of the kit of parts preaddressed or still to be addressed unless in contradiction, the kit comprises at least one of: at least two stop members which differ at least in their length along the y-axis, at least two implants having thermoplastic properties of different sizes, and a combination thereof.

The at least two stop members of this embodiment differ at least in their length along the y-axis when the stop members are attached to the ultrasound generating device.

In an embodiment of the kit of parts according to the present invention, which may be combined with any of the embodiments of the kit of parts preaddressed or still to be addressed unless in contradiction, the kit further comprises a sleeve suitable for being slidably positioned around the sonotrode along the y-axis, wherein the sleeve has a distal and a proximal end, and is configured to stop a movement of the device in y-axis direction by generating a mechanical resistance at its distal end against a surface that is essentially perpendicular to the y-axis and at its proximal end against the stop member.

As explained above, the y-axis refers to the y-axis of the device assembled from the parts of the kit of parts.

In an embodiment of the kit of parts according to the present invention, which may be combined with any of the embodiments of the kit of parts preaddressed or still to be addressed unless in contradiction, the sonotrode is the sonotrode for fixating an implant having thermoplastic properties in a recess of an object as described herein.

In an embodiment of the kit of parts according to the present invention, which may be combined with any of the embodiments of the kit of parts preaddressed or still to be addressed unless in contradiction, the suture anchor has a suture conduit at a distal end of the anchor.

In an embodiment of the kit of parts according to the present invention, which may be combined with any of the embodiments of the kit of parts preaddressed or still to be addressed unless in contradiction, the suture anchor has a suture conduit at a distal end of the anchor, and wherein the kit of parts further comprises a suture suitable for being threaded through the suture conduit or being threaded through the suture conduit.

In a further aspect, the present invention is directed to a method for fixating an implant, optionally a suture with a suture anchor, in a recess of an object, optionally at a predefined depth in the object, comprising the steps of: providing the device for fixating an implant having thermoplastic properties in a recess of an object as described herein,
providing an implant having thermoplastic properties, optionally a pin or suture anchor, optionally a suture anchor with a suture,
attaching the implant to the distal end of the sonotrode, and providing an object with a recess, wherein the recess has a cross-section corresponding to the cross-section of the implant, or has a cross-section that is smaller than the cross-section of the implant to the extent that an insertion of the implant is possible, or
has a cross-section corresponding to or being larger than the cross-section of the implant and has a depth that is shorter than the length of the implant in y-direction, followed by
inserting the implant into the recess while or followed by transmitting ultrasound from the sonotrode to the implant for a time sufficient to liquify the implant to the extent that liquefaction results in fixation of the implant in the recess, and until the stop member stops a movement of the device in y-axis direction by generating a mechanical resistance against a surface around the recess of the object.

For example, if ultrasound is applied after physical insertion of the implant into the recess, the stop member may be dimensioned such that it is not yet in contact with the surface around the recess after physical insertion but will come into contact once the ultrasound is applied because the implant “melts” into the recess.

The definitions provided above in the context of the sonotrode, the apparatus, the kit of parts, the method for assembling a device for fixating at least one implant having thermoplastic properties in a recess of an object, the device for fixating an implant having thermoplastic properties in a recess of an object, the method for fixating at least one pin or at least one suture with at least one suture anchor in a recess of an object and the kit of parts apply to all of the following method aspects and embodiments, unless specifically stated otherwise.

The implant is an implant as described above, e.g. a suture anchor that has a suture conduit at a distal end of the anchor.

The recess in the object, into which the implant is fixated, can be an elongated recess, e.g. as obtained by drilling with a drill, for example a surgical drill as explained above. The step of providing an object can be performed at any time before the step of inserting the implant (the order of the first four method steps can be changed to any order). The recess in the object may be obtained by drilling into the object with a drill of a suitable diameter (i.e. cross-section), e.g. a diameter corresponding to the cross-section of the at least one implant or having a cross-section that is larger or smaller than the cross-section of the at least one implant. In particular, the recess can be drilled such or the implant can be chosen such relative to the drilled recess that the depth of the recess (how far the recess is drilled into the object) is shorter than the length of the implant. The length of the implant refers to the distance between end points of the implant in direction of insertion into the recess, which typically corresponds to the direction along the axis of attachment of the implant to the sonotrode (tip).

In a further aspect, the present invention is directed to a method for fixating an implant, optionally a pin or suture anchor, optionally a suture with a suture anchor, in a recess of an object, optionally at a predefined depth in the object, the method comprising the steps of:
providing the device for fixating an implant having thermoplastic properties in a recess of an object as described herein,
providing an implant having thermoplastic properties, optionally a pin or suture anchor, optionally a suture anchor with a suture,
attaching the implant to the distal end of the sonotrode, providing an object with a recess, wherein the recess has a cross-section corresponding to the cross-section of the implant, or has a cross-section that is smaller than the cross-section of the implant to the extent that an insertion of the implant is possible, or
has a cross-section corresponding to or being larger than the cross-section of the implant and has a depth that is shorter than the length of the implant in y-direction,
providing a sleeve having a distal and a proximal end, positioning the sleeve with its distal end against a surface around the recess of the object, and
inserting the sonotrode in the sleeve,
followed by
inserting the implant into the recess while or followed by transmitting ultrasound from the sonotrode to the implant for a time sufficient to liquify the implant to the extent that liquefaction results in fixation of the implant in the recess, and until the stop member stops a movement of the device in y-axis direction by generating a mechanical resistance against the proximal end of the sleeve.

For the step of positioning the sleeve with its distal end against a surface around the recess of the object, it is alternatively possible to position the sleeve against a different surface than the surface of the object. For example, it may be positioned against an overlying surface, e.g. the skin, which is then used to define an implantation depth in an underlying object.

The interaction, i.e. the stop force, between the sleeve (its proximal end) and the stop member defines the insertion depth of the implant.

In an embodiment of the method for fixating an implant in a recess of an object according to the present invention, which may be combined with any of the embodiments of the method preaddressed or still to be addressed unless in contradiction, the method comprises
providing a suture anchor as an implant, wherein the suture anchor comprises a suture in a conduit at a distal end of the anchor or wherein the method comprises providing a suture anchor as an implant and threading a suture through a conduit at a distal end of the suture anchor,
inserting the suture anchor into the recess of the object while or followed by transmitting ultrasound from the sonotrode to the suture anchor for a time sufficient to liquify the suture anchor to the extent that liquefaction results in fixation of the suture by means of the anchor in the recess, and until the stop member stops a movement of the device in y-axis direction by generating a mechanical resistance against a surface around the recess of the object or against the proximal end of the sleeve.

In a further aspect, the present invention is directed to a method for fixating an implant, optionally a pin or suture anchor, optionally a suture with a suture anchor in a recess of an object at a predefined depth in the recess, the method comprising the steps of:
(a) providing an ultrasound generating device having a sonotrode attached to a distal end of the ultrasound generating device at a proximal end of the sonotrode, wherein the sonotrode has a distal end configured to receive an implant having thermoplastic properties, optionally a pin or suture anchor, and has a y-axis extending from the proximal end to the distal end of the sonotrode,
(b) providing at least one stop member and attaching it to the ultrasound generating device, wherein the at least one stop member is isolated against ultrasound, wherein the at least one stop member at least partially or fully encloses the sonotrode around the y-axis, and wherein the at least one stop member is shorter than the sonotrode in y-axis direction from the proximal to the distal end of the sonotrode,
(c) providing at least one implant having thermoplastic properties, optionally a pin or suture anchor, optionally a suture anchor with a suture, and attaching it to the distal end of the sonotrode, and
(d) providing an object with a recess, wherein the recess has a cross-section corresponding to the cross-section of the implant, optionally pin or suture anchor, or has a cross-section that is smaller than the cross-section of the implant, optionally pin or suture anchor, to the extent that an insertion of the implant, optionally pin or suture anchor, is possible, or
has a cross-section corresponding to or being larger than the cross-section of the implant, optionally pin or suture anchor, and has a depth that is shorter than the length of the implant, optionally pin or suture anchor, in y-direction, followed by
(f) inserting the implant, optionally pin or suture anchor, into the recess while or followed by transmitting ultrasound from the sonotrode to the implant, optionally pin or suture anchor, for a time sufficient to liquify the implant, optionally pin or suture anchor, to the extent that liquification results in fixation of the implant, optionally a suture by means of the anchor, in the recess, and until the at least one stop member stops a movement of the device in y-axis direction by generating a mechanical resistance against a surface around the recess of the object.

In step (c) a suture anchor with a suture can be provided, or alternatively, a suture anchor can be provided and the suture can be provided and/or attached to the suture anchor in a separate step before inserting the suture anchor into the recess.

In a further aspect, the present invention is directed to a method for fixating an implant, optionally a pin or suture anchor, optionally a suture with a suture anchor, in a recess of an object, optionally at a predefined depth in the recess, the method comprising the steps of:
(a) providing an ultrasound generating device having a sonotrode attached to a distal end of the ultrasound generating device at a proximal end of the sonotrode, wherein the sonotrode has a distal end configured to receive an implant having thermoplastic properties, optionally a pin or a suture anchor, and has a y-axis extending from the proximal end to the distal end of the sonotrode,
(b) providing at least one stop member and attaching it to the ultrasound generating device, wherein the at least one stop member is isolated against ultrasound, wherein the at least one stop member is shorter than the sonotrode in y-axis direction from the proximal to the distal end of the sonotrode,
(c) providing at least one implant having thermoplastic properties, optionally a pin or suture anchor, and attaching it to the distal end of the sonotrode, and
(d) providing an object with a recess, wherein the recess has a cross-section corresponding to the cross-section of the implant, optionally pin or suture anchor, or has a cross-section that is smaller than the cross-section of the implant, optionally pin or suture anchor to the extent that an insertion of the implant, optionally pin or suture anchor, is possible, or has a cross-section corresponding to or being larger than the cross-section of the implant, optionally pin or suture anchor, and has a depth that is shorter than the length of the implant, optionally pin or suture anchor, in y-direction,
(e1) providing a sleeve having a distal and a proximal end,
(e2) positioning the sleeve with its distal end against a surface around the recess of the object, and
(e3) inserting the sonotrode in the sleeve, followed by
(f) inserting the implant, optionally pin or suture anchor, into the recess while or followed by transmitting ultrasound from the sonotrode to the implant, optionally pin or suture anchor, for a time sufficient to liquify the implant, optionally pin or suture anchor, to the extent that liquification results in fixation of the implant, optionally pin or suture anchor, optionally suture, by means of the implant, optionally pin or suture anchor in the recess, and until the at least one stop member stops a movement of the device in y-axis direction by generating a mechanical resistance against the proximal end of the sleeve.

In step (c) a suture anchor with a suture can be provided, or alternatively, a suture anchor can be provided and the suture can be provided and/or attached to the suture anchor in a separate step before inserting the suture anchor into the recess.

In an embodiment of the method for fixating an implant, optionally a pin or suture anchor, optionally a suture with a suture anchor, in a recess of an object, optionally at a predefined depth in the recess, according to the present invention, which may be combined with any of the embodiments of the method preaddressed or still to be addressed unless in contradiction, after step (f), the method further comprises the following steps:
removing the at least one stop member of previous step (b), providing a second stop member and attaching it to the ultrasound generating device, wherein the second stop member is isolated against ultrasound, wherein the second stop member is shorter than the sonotrode in y-axis direction from the proximal to the distal end of the sonotrode, and wherein the second stop member is of a different length in y-axis direction compared to the at least one stop member of previous step (b), and repeating steps (c) to (f).

The step of removing the at least one stop member of previous step (b) and attaching a second stop member means that the stop member is detached from/attached to the device, in particular from/to the ultrasound generating device. For this embodiment of the method, the ultrasound generating device used is, e.g., detachably coupled to the sonotrode, e.g. by a plug-fit connection, a clip-on connection, a screw connection or a bayonet mechanism. The use of differently sized stop members (e.g. of different lengths in y-direction) allows for the implantation of differently sized implants, implantation at different depths in the recess in the object, and/or the use of a sleeve or no sleeve. Of course, the sleeve can be exchanged additionally or alternatively for a differently sized sleeve. It is also possible to exchange the stop member and the sleeve together for a stop member and sleeve of different dimensions (in particular of a different length in y-direction) in order to account for differently sized implants and/or different implant depths.

In an embodiment of the method for fixating an implant, optionally a pin or suture anchor, optionally a suture with a suture anchor, in a recess of an object, optionally at a predefined depth in the recess, according to the present invention, which may be combined with any of the embodiments of the method preaddressed or still to be addressed unless in contradiction, after step (f), the method comprises the following steps:

providing a further implant having thermoplastic properties, optionally a further suture anchor, and attaching it to the distal end of the sonotrode, wherein the further implant, optionally the further suture anchor, is of a different size compared to the at least implant, optionally the at least one suture anchor, and repeating steps (d) to (f).

When a differently sized implant, optionally suture anchor, is used, the stop member and/or sleeve may be exchanged for a stop member and/or sleeve of different dimensions, in particular of a different length in y-direction.

For example, and for all method aspects disclosed herein, the implant, optionally pin or suture anchor, is inserted into a recess of an object such that the implant is flush with the surface of the object, or such that there is a distance (termed “implant depth” or “implantation depth”) between the surface around the recess of the object and a proximal end of the implant, optionally pin or suture anchor, of about 0.25 to 1.5 mm, optionally about 0.5 mm to 1 mm. This implant depth is controlled by the stop member and/or sleeve used for the method. In an embodiment of the method for fixating an implant, optionally a pin or suture anchor, optionally a suture with a suture anchor, in a recess of an object, optionally at a predefined depth in the recess, according to the present invention, which may be combined with any of the embodiments of the method preaddressed or still to be addressed unless in contradiction, the object is a human, animal or artificial hard tissue, a cartilage-covered hard tissue, or an implant structure, optionally the hard tissue is a hard tissue sample outside of the human or animal body, a hard tissue of a dead body or a hard tissue of a human or animal patient, and the implant structure optionally is a fixation plate or bone plate.

In an embodiment of the method for fixating an implant, optionally a pin or suture anchor, optionally a suture with a suture anchor, in a recess of an object, optionally at a predefined depth in the recess, according to the present invention, which may be combined with any of the embodiments of the method preaddressed or still to be addressed unless in contradiction, the sonotrode is the sonotrode for fixating an implant having thermoplastic properties in a recess of an object as described herein.

### BRIEF DESCRIPTION OF THE DRAWINGS

Embodiments of the current invention are described in more detail in the following with reference to the figures. These are for illustrative purposes only and are not to be construed as limiting. It is shown in:

FIG. 1A, FIG. 1B and FIG. 1C side and cut views of a sonotrode according to an embodiment;

FIG. 2A and FIG. 2B side views of sonotrodes according to an embodiment;

FIG. 3 a side view of a sonotrode of an embodiment and an angled view of corresponding implants;

FIG. 4A and FIG. 4B cut views of sonotrodes with plugged-on implants of an embodiment;

FIG. 5 a side view of sonotrodes with plugged-on implants of an embodiment;

FIG. 6A, FIG. 6B and FIG. 6C sonotrodes not according to the present invention;

FIG. 7A, FIG. 7B, FIG. 7C, FIG. 7D and FIG. 7E side views of devices according to an embodiment;

FIG. 8A and FIG. 8B side views of devices according to an embodiment and a stop member;

FIG. 9A and FIG. 9B side views of devices according to an embodiment;

FIG. 10 an illustration of implant depth;

FIG. 11A and FIG. 11B side views of a device according to an embodiment and a sleeve;

FIG. 12 a side view of different sleeves for the device of an embodiment;

FIG. 13 a side view of different sleeves for the device of an embodiment;

FIG. 14A and FIG. 14B side views of devices of an embodiment with different surfaces;

FIG. 15A and FIG. 15B side views of different sleeves for the device of an embodiment; and

FIG. 16A, FIG. 16B, FIG. 16C and FIG. 16D illustrations of recess dimensions.

### DETAILED DESCRIPTION

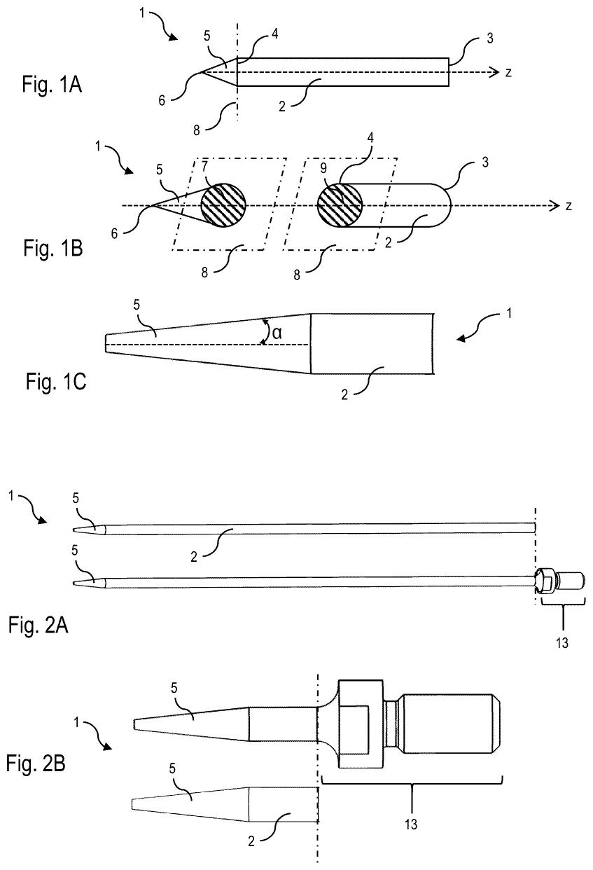

FIG. 1A shows a side view of a sonotrode 1 according to the present invention. In this embodiment, the sonotrode features an elongated body 2 with a proximal end 3 and a tapered tip 5 at the distal end 4 of the body. An imaginary z-axis leads from the distal end 6 of the tip with to the proximal end 4 of the body. Independent of the embodiment shown, the z-axis can be described as a longitudinal axis. Normal to the z-axis a plane 8 is defined. As also shown in FIG. 1B, the area 7 of the tapered tip at the distal end 4 of the body projected onto this plane 8 fully covers the area 9 of the distal end 4 of the body projected onto the plane 8. The area 7 of the tapered tip may also be larger than the area 9 of the body, but not smaller. FIG. 1B shows a detailed cross-sectional view of the area 7 of the tapered tip at the distal end 4 of the body projected onto the plane 8, which fully covers the area 9 of the distal end 4 of the body projected onto the plane 8. If the tapered tip and/or the body were to comprise hollow sections, these hollow sections would also be considered for (i.e. “counted as”) the areas 7 and 9, respectively. The areas 7 and 9 can also be considered cross sections, and, for cylindrical embodiments of the tip 5 and body 2, the largest circumference or diameter of the tip (i.e. where it meets the body) is equal to or greater than the circumference or diameter of the body at its distal end 4. The body 2 may be cylindrical or prismatic or have any other shape as long as the areas of the tip 7 and the body 9 are as described relative to one another. The tapered tip 5 is shown with a pointy tip, but may feature a flattened tip. The relative “length” of the tip 5 and the body 2 in direction of the z-axis are not limited and any ratio is within the scope of the present invention. The sonotrode 1 may also consist entirely of the tapered tip 5, i.e. the body 2 can be part of the tip 5. Although the tip 5 and body 2 are drawn as separate entities, these may also be one unit, e.g. a monobloc, or may be reversibly separable. The tapered tip is tapered from the body 2 to the tip 6 and may be a conus or have any other, e.g. polygonal, shape. FIG. 1C shows the tapering angle x of the tapered tip 5.

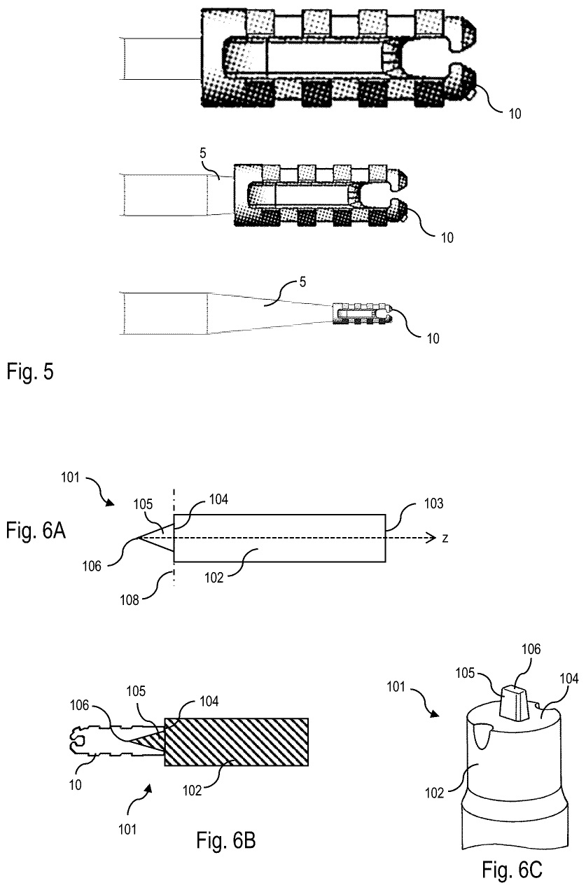

FIG. 2A and FIG. 2B show embodiments of a sonotrode 1 according to the present invention. In these embodiments, the sonotrode 1 has longer (elongated in z-direction) (FIG. 2A) and shorter (FIG. 2B) body 2. For example, the sonotrode may have an overall length in z-direction (left to right in the Figure) excluding attachment 13 of 50 mm to 300 mm (“longer version” FIG. 2A) or 5 mm to 25 mm (“shorter version” FIG. 2B), optionally about 14 mm or 118 mm. Optionally, the length of the sonotrode along the z-axis can also correspond to the formula

$$l = n * ( \frac{λ}{2} ) ,$$

wherein l is the length of the sonotrode, n is an integer from 1 to 6 and A is the resonance length of the sonotrode at the ultrasound frequency generated by the ultrasound generating device in kHz, optionally at a frequency from 15 kHz to 45 kHz, optionally 20 kHz to 30 kHz. For example, the diameter of the sonotrode body 2 may be 1 mm to 6 mm, optionally about 2.3 mm. The tapered tip 5 may have a tapering angle of about 1 to 20 degrees, optionally about 6 degrees, and the tip may have a smallest diameter of about 0.1 to 4 mm, optionally about 0.65 mm. The longer body may be used, e.g. in minimally invasive surgery. At the proximal end 3 of the body, an attachment 13 may be located. This attachment 13 may be a coupling means for coupling the sonotrode to an ultrasound generating device or it may be an ultrasound generating device. FIG. 2A and FIG. 2B show that the relation of the areas (or cross sections) 7 and 9 described above applies to where the tip and the body “meet”, i.e. to the area where the tapering ends and the body starts (its distal end). The areas further away from this point, e.g. where attachment 13 is located, are not limited by the definition of the relation between the areas 7 and 9.

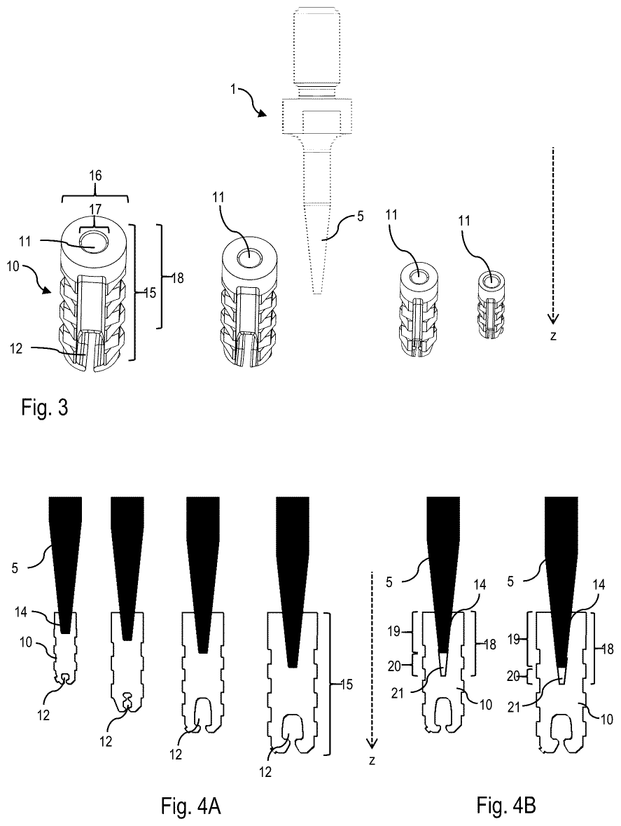

FIG. 3 shows a sonotrode 1 according to the present invention and implants, e.g. suture anchors, 10 of different size with a suture conduit 12. The implant 10 can be plugged onto the sonotrode tip 5 via its cavity 11. The cavity (or recess) 11 of the implant is preferably elongated in z-direction. Due to the shape of the sonotrode, the tapered tip 5 will exclusively be in contact with the cavity 11 in the implant 10. Also, different sizes of implants with the same or differently sized cavities 11 can be attached to the tip 5 of the same sonotrode 1. The sonotrode shown in this figure is exemplary only and can be any sonotrode described herein, e.g. one described in FIG. 1A, FIG. 1B, FIG. 1C, FIG. 2A or FIG. 2B. The suture anchor 10 may be an essentially cylindrical structure with a length 15 of 4 to 20 mm, an outer diameter 16 of 1 to 9 mm, and with a cavity length 18 of 0.5 to 8 mm and having a cavity diameter 17 of 0.2 to 8 mm. The cavity 11 may be cylindrical or have a conus (tapered) shape (with the largest diameter at the proximal end of the suture anchor). The suture anchor may also be a pin, e.g. lacking the suture conduit, as described herein and may have the same dimensions as the anchor or have the following dimensions: Diameter between 1 mm and 10 mm, length between 4 mm and 60 mm, the pin can also have a head to create a positive fit with a bone surface or a plate hole. The ultrasound is only transferred via the surface of the cavity, and the proximal surface of the pin can be convex or concave, it can comprise functional geometries, such as slots or geometries such as inner or outer torx that would allow to remove or to attach other elements. The outer circumferential surface of the suture anchor or pin may be irregular and/or, e.g., feature protrusions as shown in the figure, e.g. as detailed in WO 2012/037699, or it may be essentially even. As shown in FIG. 3, the pin or suture anchor can have an essentially flat surface at its proximal end, e.g. a flat surface perpendicular to the axis of tip insertion (along the z-axis), which axis can correspond to the direction of elongation of the suture anchor or pin and its cavity. In other words, the suture anchor or pin at its proximal end can, for example, have a shape that corresponds to a shape that would result from cutting through the suture anchor or pin essentially perpendicular to the axis of tip insertion (along the z-axis).

FIG. 4A and FIG. 4B show cut views of the same sonotrode with the same sonotrode tip 5 and differently sized implants 10 (from left to right in each FIG. 4A and FIG. 4B) plugged onto the sonotrode tip. As shown in FIG. 4A and FIG. 4B, the implant 10 may be a suture anchor and may feature a suture conduit 12 at the distal end of the anchor. As can be seen, the same sonotrode is suitable for receiving differently sized implants due to the shape of the sonotrode. The area of contact 14 between the sonotrode and the implant is located exclusively in the cavity of the implant and no other parts of the implant are in physical contact with the sonotrode. Vibrational energy is at least partially transmitted from the sonotrode to the implant via the area of contact 14; for example, not a full amplitude may be transmitted from the sonotrode to the implant but rather the ultrasonic impulse is transmitted to the implant. In any case, transmission is such that the implant having thermoplastic properties is at least partially liquified by the ultrasound transmitted from the sonotrode, e.g. to the extent that fixation of the implant in a recess of an object is achieved after solidification of the implant. FIG. 4B details that the implant (e.g. the suture anchor or pin) can, e.g., feature a cavity that is dimensioned such that a distal end of the sonotrode tip 5 does not reach the end of the cavity (in z-axis direction) in the absence of ultrasound. In other words, the cavity depth or length 18 can be defined relative to the sonotrode tip- or vice-versa—in that the cavity is longer, e.g. about 5% to 50% longer, in z-direction than the length of the part 19 of the sonotrode tip that inserts into the cavity, i.e. the insertion depth, in the absence of ultrasound generation. In other words, distance 18 can be longer, e.g. 5% to 50% longer, than distance 19. This leaves a free distance 20 in the cavity and a free cavity volume 21. For example, the cavity can be clear of the sonotrode tip (in the absence of ultrasound) for a distance 20 of about 0.1 to 5 mm measured from the distal end of the sonotrode tip inserted into the cavity to the end of the cavity in y-axis direction before ultrasound is generated. The geometry of the sonotrode tip 5 and the cavity in the implant can allow that upon ultrasound generation, the tip 5 can insert a bit further into the implant's cavity, thus increasing the area of contact 14 and improving liquification of the implant.

FIG. 5 shows three side views of the same sonotrode with the same sonotrode tip 5 and three differently sized implants 10 (from top to bottom) plugged onto the sonotrode tip, in analogy to FIG. 4A and FIG. 4B. As can be seen, the same sonotrode is suitable for receiving differently sized implants due to the shape of the sonotrode and no other parts of the implant other than its cavity are in physical contact with the sonotrode.

FIG. 6A, FIG. 6B and FIG. 6C show different views of a sonotrode that is not according to the present invention. FIG. 6A shows that—in contrast to the sonotrode 1 of the present invention-a sonotrode 101 not according to the present invention with a tapered tip 105, a distal end of the tip 106, a body 102 and a proximal end of the body 103. The sonotrode 101 features an area of the tapered tip at the distal end 104 of the body that, when projected onto a plane 108 perpendicular to the z-axis, does not fully cover the area of the distal end 104 of the body projected onto the plane 108. The area of the tapered tip is smaller than the area of the body, and not equal or larger. FIG. 6B shows an implant 10 plugged onto the tip 105 of sonotrode 101. Due to the larger area of the distal end 104 of the body, the circumference around the cavity of the implant 10 will additionally be in physical contact with the sonotrode 101, which is in contrast to the sonotrode 1 of the present invention. This means that sonotrode 101 can only be used for implants of specific sizes.

Furthermore, the geometry of sonotrode 101 can cause contact of the vibrating sonotrode (the part of the area of the body that is larger than the area of the tip), e.g. with sensitive tissue (e.g. cartilage) located around or on top of the location (e.g. a recess or opening in a hard tissue such as a bone) where the implant is fixated, which can cause damage to the tissue. FIG. 6C shows another example of a sonotrode 101 not according to the present invention which features an area of the tapered tip 105 at the distal end 104 of the body that does not fully cover the area of the distal end 104 of the body 102.

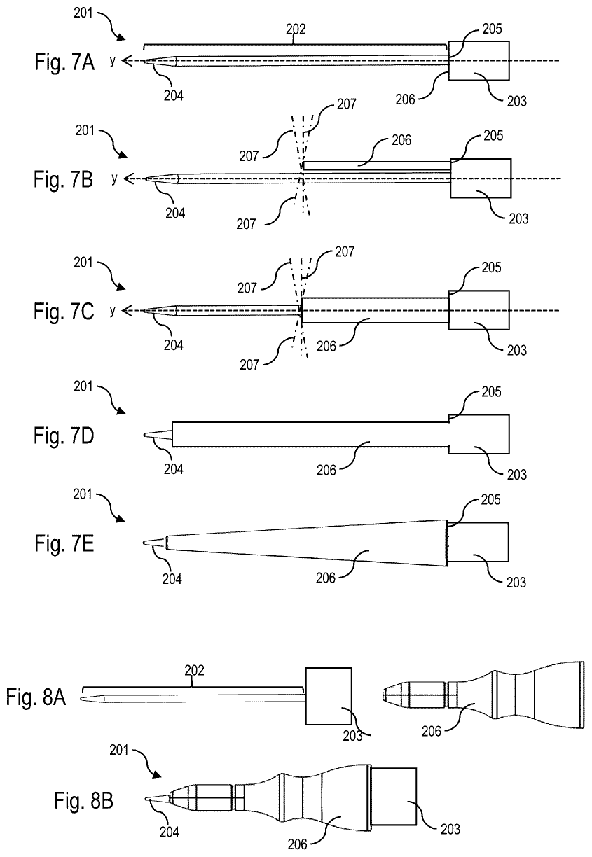

FIG. 7A, FIG. 7B, FIG. 7C, FIG. 7D and FIG. 7E show different embodiments of the device 201 of the present invention. In FIG. 7A, an exemplary device 201 is shown with a sonotrode 202 having a distal end 204, which can be in the form of a tapered tip. The ultrasound generating device 203 is functionally connected to the sonotrode at its distal end 205. The proximal end of the sonotrode is the end that is connected to the ultrasound generating device 205. The y-axis is an axis extending from the proximal end to the distal end 204 of the sonotrode 202. In this embodiment, the stop member 206 is part of the ultrasound generating device, for example it is a surface facing the sonotrode and being essentially orthogonal to the y-axis. The stop member 206 is isolated against the ultrasound generated by the ultrasound generating device and against the ultrasound (vibrational energy) transmitted by the sonotrode. In FIG. 7B, a different embodiment is shown, wherein the stop member 206 extends in y-axis direction along the sonotrode. The stop member 206 can generate a mechanical resistance against a surface 207 that is essentially perpendicular to the y-axis and will stop movement of the device 201 in y-direction. The term “essentially perpendicular to the y-axis” includes but is not limited to an angle of about 90° to the y-axis but also includes angles of about 15° to 165°. The stop member is made of a material that is dimensionally stable to the extent that it can withstand manual pressure of a person operating the device against the surface 207 without being significantly deformed, which means that there is no deformation that would allow for significant movement of the device in y-direction when the stop member meets surface 207. The mechanical resistance can be generated, e.g. by a distal end (e.g. a surface) of the stop member 206, with its the proximal end located at or near the ultrasound generating device 203. The stop member 206 may enclose (partially or fully) the sonotrode 202 around the y-axis as shown in FIG. 7C and FIG. 7D. As shown in FIG. 7E, the stop member 206 can have different shapes including a cylindrical or prismatic (e.g. polygonal) shape, which may be tapered, and it can be dimensioned such that it can be positioned around the sonotrode (around the y-axis), in particular such that it has no physical contact with the sonotrode to avoid the transmission of vibrational energy from the sonotrode to the sleeve. The stop member can also be tapered or irregular in shape, however, and as shown in FIG. 7A to 7E, the stop member is shorter than the sonotrode in y-axis direction from the proximal to the distal end 204 of the sonotrode (i.e. the sonotrode 202 or at least its distal end 204 extends further in y-axis direction than the stop member 206). FIG. 8A and FIG. 8B show a stop member 206 detached from the ultrasound generating device 203. In FIG. 8B, the stop member 206 is attached to the ultrasound generating device 203, thus forming device 201. The stop member 206 shown in this figure can, for example, generate a mechanical resistance at its distal end facing the distal end 204 of the sonotrode 202, while being attached to the ultrasound generating device at its proximal end.

FIG. 9A and FIG. 9B show embodiments of the device 201 according to the present invention with an implant 210 plugged onto the distal end 204 of the sonotrode. The distance between the distal end of the stop member 206 and the proximal end of the implant 210 defines the implant depth 208. The implant depth 208 is defined by one or more of: the implant size (its elongation in y-direction), the sonotrode's distal end 204 (e.g. how far the distal end 204 inserts into the implant 210) and/or the length of the stop member 206 in y-direction.

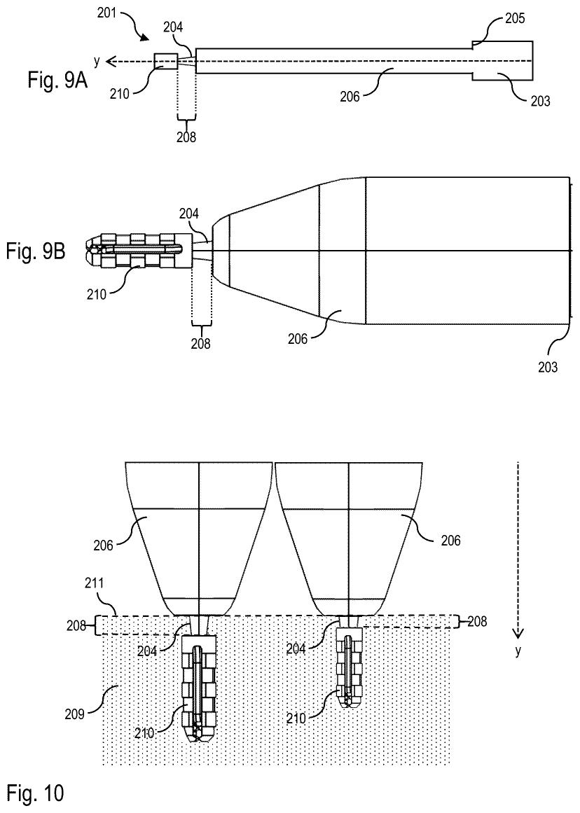

FIG. 10 illustrates the implant depths 208 of two implants 210 in an object 209 (e.g. a tissue, in particular a hard tissue, in particular a bone). The stop member 206 generates a mechanical resistance against the surface 211 of the object 209 around the recess into which the implant 210 is inserted. The distance between the distal end of the stop member 206 and the proximal end of the implant 210 defines the implant depth 208. The implant depth 208 can be adjusted, e.g., depending on the implant size (left side: larger implant, right side: smaller implant). Smaller implants (right side) can, for example, be implanted at a smaller depth than larger implants (left side) while using the same device (the same sonotrode 202 (only distal end 204 shown) and the same ultrasound generating device 203 (not shown)) with differently dimensioned stop members 206. The stop members 206 can be different in their length along the y-axis which translates into different implant depths 208. Exemplary implant depths 208 include about 0.25 to 1.5 mm, optionally about 0.5 mm to 1 mm.

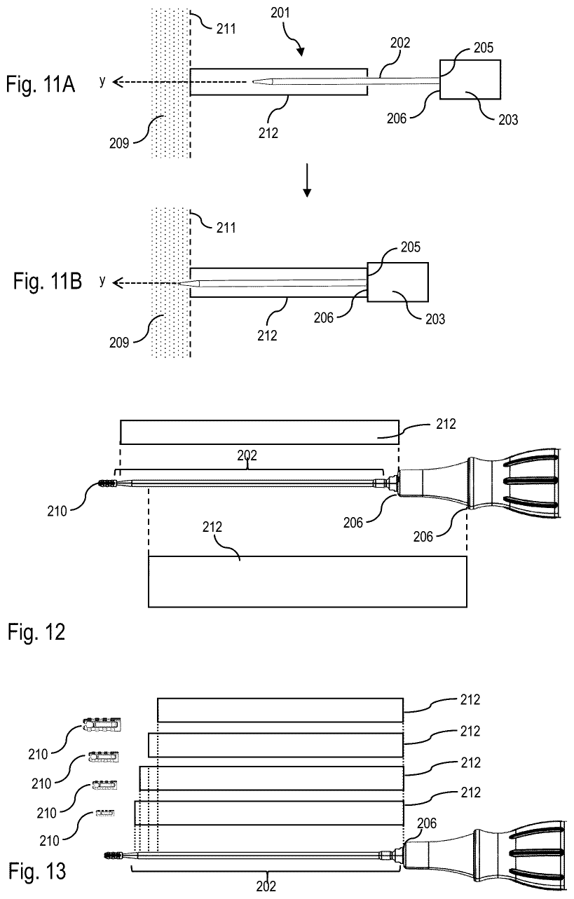

FIG. 11A and FIG. 11B illustrate an embodiment of the device 201 according to the present invention, in which the device further comprises a sleeve 212 slidably positioned around the sonotrode 202 along the y-axis. FIG. 11A shows how the distal end of the sleeve is positioned on the surface 211 of an object 209, thus generating a mechanical resistance, e.g. a stop, against this surface. When the sonotrode 202 and ultrasound generating device 203 are moved in y-direction to insert an implant into a recess in the object 209, the proximal end of the sleeve generates a mechanical resistance against the stop member 206 when the proximal end and the stop member 206 “meet”, as shown in FIG. 11B. As can be seen in this figure, the length of the sleeve 212 along the y-axis, the extension of the stop member 206 along the y-axis, or both can be used to determine the implant depth. Of course, the sleeve 212 can also be used in minimally invasive surgery, where the sleeve would be inserted through a first surface (e.g. the skin of a patient) and would then be positioned to generate a mechanical resistance at its distal end against a second surface of the object into which an implant is inserted, e.g. a bone. The sleeve 212 of all aspects and embodiments disclosed herein is dimensionally stable, at least in y-axis direction, and can be made of a material as explained for the stop member 206. The sleeve 212 may also be flexible or bendable to the extent that it is stable enough to set a distance between the surface 211 around the recess in the object and the stop member.

FIG. 12 shows how differently dimensioned sleeves 212 can be used with a device featuring different stop members 206. For example, the stop member 206 can generate a mechanical resistance against the proximal end of the sleeve 212 at different positions (labelled 206 in the Figure), e.g. depending on the shape of the sleeve 212 that is used.

FIG. 13 illustrates how differently dimensioned sleeves 212 can be used in combination with differently sized implants 210 on the same sonotrode 202 and ultrasound generating device with the same stop member 206. For example, a comparatively short sleeve 212 can be used with a comparatively large implant (topmost sleeve/implant) to achieve a larger implant depth compared to a longer sleeve (e.g. bottom sleeve) used together with a smaller implant on the same sonotrode 202 and stop member 206.

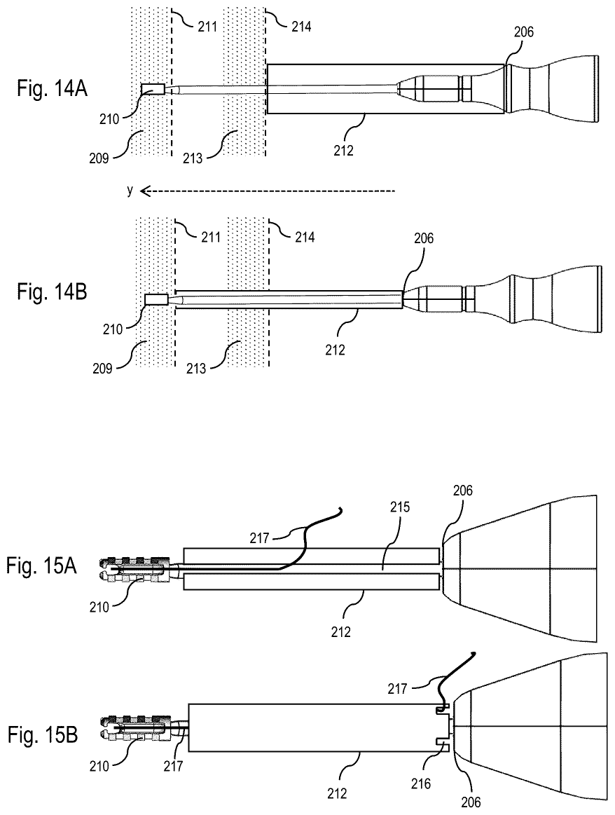

FIG. 14A and FIG. 14B illustrate two embodiments of the device 201 according to the present invention, in which the device further comprises a sleeve 212 slidably positioned around the sonotrode 202 along the y-axis. In FIG. 14A the distal end of the sleeve is positioned on a further surface 214 of a further object 213 (e.g. the skin), thus generating a mechanical resistance, e.g. a stop, against this further surface 214. The sonotrode 202 and ultrasound generating device 203 are moved in y-direction through the further surface 214 and further object 213 to reach and insert an implant 210 into a recess in the object 209 when the proximal end of the sleeve 212 generates a mechanical resistance against the stop member 206. In this example, the implant depth in the object 209 is defined relative to the further surface 214 by the dimension (length along the y-axis) of the sleeve and/or the stop member 206. The position of the stop member 206 is not limited to the location shown in FIG. 14A, i.e. the stop member of FIG. 14A may also be the stop member shown in FIG. 14B- and vice-versa- or at any other location that is connected to the ultrasound generating device. The embodiment of FIG. 14B shows a sleeve 212 that is inserted through the further surface 214 and through the further object 213 (e.g. the skin), for example in the context of minimally invasive surgery. The distal end of the sleeve is positioned on a surface 211 of an object 209 (e.g. a bone), thus generating a mechanical resistance, e.g. a stop, against this surface 211. The sonotrode 202 and ultrasound generating device 203 are moved in y-direction through the further surface 214 and further object 213 to reach and insert an implant 210 into a recess in the object 209 when the proximal end of the sleeve generates a mechanical resistance against the stop member 206.

FIG. 15A and FIG. 15B show different embodiments of sleeves 212 of the device according to the present invention. For example, when the present device is used for implanting a suture anchor 210 and for fixating a suture 217 in an object 209 (e.g. a bone) with the suture anchor, the suture 217 should have no impact on the implant depth. If the suture 217 were positioned between a surface against which the sleeve 212 forms a mechanical resistance, e.g. surfaces 211 or 214, the suture 217 would change the distance between the surface (e.g. 211 or 214) and the distal end of the sleeve 212, and thus have an impact on the implant depth. The same is true if the suture 217 were located between the proximal end of the sleeve 212 and the stop member 206, so that it would impact the surfaces of the sleeve and the stop member “meeting” one another. In order to avoid any impact of the suture 217 on the implant depth, the sleeve 212 according to any aspect and embodiment of the present invention, and independent from FIG. 15A and FIG. 15B, may comprise means for guiding the suture such that the implant depth is not affected, i.e. such that the suture is not located between the surface (e.g. 211 or 214) against which the sleeve 212 generates a resistance and the sleeve 212, and not located between the sleeve 212 and the stop member 206 such that it would affect the distance between the sleeve 212 and the stop member 206. These means can, for example, be at least one slot 215 in the sleeve, either a slot 215 extending along the entire sleeve (FIG. 15A) or at least one slot at the distal end of the sleeve, through which the suture 217 is guided. Also, the means can be a slot at the proximal end, e.g. in the form of one or more recesses 216 (FIG. 15B) through which the suture can be guided. Of course, the one or more recesses 216 could alternatively be located on the stop member 206 to achieve the same effect.

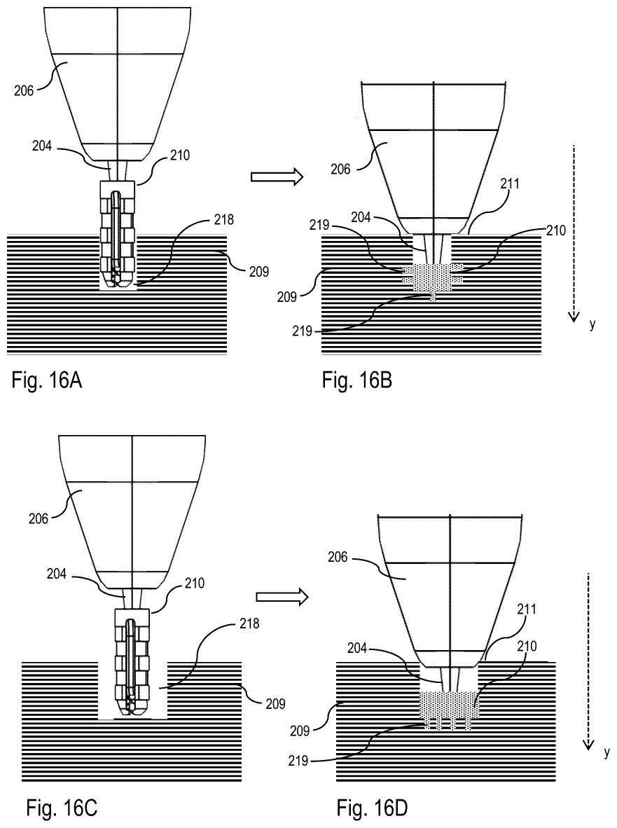

FIG. 16A, FIG. 16B, FIG. 16C and FIG. 16D show implantation of implants 210 in two differently dimensioned recesses 218. In FIG. 16A the recess 218 has a cross-section corresponding to the cross-section of the implant 210 and has a depth that is shorter than the length of the implant in y-direction. After insertion of the implant 210 ultrasound is applied and the distal end of the sonotrode 204 and the stop member 206 are pushed further in y-direction as the implant 210 melts in the recess, as shown in FIG. 16B. The distal end of the sonotrode 204 is stopped in its movement by the stop member 206 making physical contact with the surface 211 of the object 209. FIG. 16C and FIG. 16D show the same scenario for a recess 218 with a cross-section larger than the cross-section of the implant 210 and a depth that is shorter than the length of the implant in y-direction. In both FIG. 16B and FIG. 16D, an optional flow or extension of the liquified implant into the bone structure 219 around the recess is shown schematically. An extension of the liquified implant can occur or typically occurs, e.g., due to the porous nature of the bone structure. As outlined above, the flow of the implant into the bone structure can be controlled by the relative cross-section of the recess and the implant, e.g. such that it flows into bone areas of greater stability which will improve fixation of the implant or, e.g., of a suture with the implant. In the example of FIG. 16B, the implant cross-section and the diameter of the cross-section are corresponding in size and a part of the liquified implant can “flow into”, i.e. extend into, the bone structure at the wall of the recess (and optionally, e.g. to a lesser extent, also at the bottom of the recess). On the other hand, in the example of FIG. 16D, the cross-section of the implant is smaller than the cross-section of the recess, and the liquified implant will rather or to a greater extent extend into the bone at the bottom of the recess and to a lesser extent or not at all at the wall of the recess. The relative quantities of extensions 219 are not to size nor are they limiting and shown for illustrative purposes only.

### REFERENCE SIGNS LIST

1 Sonotrode
2 Body
3 Proximal end of body
4 Distal end of body
5 Tapered tip
6 Distal end of tip
7 Area of tapered tip at distal end of body
8 Plane normal to z-axis
9 Area of the distal end of body
10 Implant
11 Cavity or recess in implant
12 Suture conduit
13 Attachment
14 Area of contact
15 Length of suture anchor
16 Outer diameter of suture anchor
17 Cavity diameter of suture anchor
18 Length of cavity
19 Insertion depth
20 Free distance in cavity
21 Free cavity volume
101 Sonotrode (not according to the invention)
102 Body
103 Proximal end of body
104 Distal end of body
105 Tapered tip
106 Distal end of tip
108 Plane normal to z-axis
201 Device
202 Sonotrode
203 Ultrasound generating device
204 Distal end of sonotrode
205 Distal end of ultrasound generating device
206 Stop member
207 surfaces essentially perpendicular to the y-axis
208 Implant depth
209 Object (e.g. tissue)
210 Implant
211 Object surface
212 Sleeve
213 Further object
214 Further surface
215 Slot
216 Recess in stop member or sleeve
217 Thread
218 Recess in object
219 Liquified implant in bone structure

1. A method for fixating an implant in a recess of an object comprising the steps of:
providing a device for fixating an implant having thermoplastic properties in a recess of an object, the device comprising:
an ultrasound generating device;
a sonotrode attached to a distal end of the ultrasound generating device at a proximal end of the sonotrode;
a stop member attached to or being part of the ultrasound generating device and being isolated against ultrasound, wherein:
the sonotrode has the distal end configured to receive an implant having thermoplastic properties and has a y-axis extending from the proximal end to the distal end of the sonotrode,
the stop member is shorter than the sonotrode in y-axis direction from the proximal to the distal end of the sonotrode, and
the stop member is configured to stop a movement of the device in a y-axis direction by generating a mechanical resistance against a surface that is essentially perpendicular to the y-axis,
providing an implant having thermoplastic properties, attaching the implant to the distal end of the sonotrode, and providing an object with a recess, wherein the recess:
has a cross-section corresponding to or being larger than the cross-section of the implant, and
has a depth that is shorter than the length of the implant in y-direction, followed by
inserting the implant into the recess, followed by
transmitting ultrasound from the sonotrode to the implant for a time sufficient to liquify the implant to the extent that liquefaction results in fixation of the implant in the recess, and until the stop member stops a movement of the device in y-axis direction by generating a mechanical resistance against a surface around the recess of the object.

2. A method for fixating an implant in a recess of an object comprising the steps of:
providing a device for fixating an implant having thermoplastic properties in a recess of an object, the device comprising:
an ultrasound generating device,
a sonotrode attached to a distal end of the ultrasound generating device at a proximal end of the sonotrode, a stop member attached to or being part of the ultrasound generating device and being isolated against ultrasound, wherein
the sonotrode has a distal end configured to receive an implant having thermoplastic properties and has a y-axis extending from the proximal end to the distal end of the sonotrode,
the stop member is shorter than the sonotrode in y-axis direction from the proximal to the distal end of the sonotrode,
the stop member is configured to stop a movement of the device in y-axis direction by generating a mechanical resistance against a surface that is essentially perpendicular to the y-axis,
providing an implant having thermoplastic properties, attaching the implant to the distal end of the sonotrode, providing an object with a recess, wherein the recess:
has a cross-section corresponding to or being larger than the cross-section of the implant, and
has a depth that is shorter than the length of the implant in y-direction, providing a sleeve having a distal and a proximal end,
positioning the sleeve with its distal end against a surface around the recess of the object, and
inserting the sonotrode in the sleeve,
followed by
inserting the implant into the recess, followed by transmitting ultrasound from the sonotrode to the implant for a time sufficient to liquify the implant to the extent that liquefaction results in fixation of the implant in the recess, and until the stop member stops a movement of the device in y-axis direction by generating a mechanical resistance against the proximal end of the sleeve.

3. The method according to claim 1, wherein the method comprises providing a suture anchor as an implant, wherein the suture anchor comprises a suture in a conduit at a distal end of the anchor or wherein the method comprises providing a suture anchor as an implant and threading a suture through a conduit at a distal end of the suture anchor,
inserting the suture anchor into the recess of the object, followed by
transmitting ultrasound from the sonotrode to the suture anchor for a time sufficient to liquify the suture anchor to the extent that liquefaction results in fixation of the suture by means of the anchor in the recess, and until the stop member stops a movement of the device in y-axis direction by generating a mechanical resistance against a surface around the recess of the object or against the proximal end of a sleeve.

4. The method according to claim 2, wherein the method comprises providing a suture anchor as an implant, wherein the suture anchor comprises a suture in a conduit at a distal end of the anchor or wherein the method comprises providing a suture anchor as an implant and threading a suture through a conduit at a distal end of the suture anchor,
inserting the suture anchor into the recess of the object, followed by
transmitting ultrasound from the sonotrode to the suture anchor for a time sufficient to liquify the suture anchor to the extent that liquefaction results in fixation of the suture by means of the anchor in the recess, and until the stop member stops a movement of the device in y-axis direction by generating a mechanical resistance against a surface around the recess of the object or against the proximal end of the sleeve.

5. A method for fixating a suture with a suture anchor in a recess of an object comprising the steps of:
(a) providing an ultrasound generating device having a sonotrode attached to a distal end of the ultrasound generating device at a proximal end of the sonotrode, wherein the sonotrode has a distal end configured to receive a suture anchor having thermoplastic properties and has a y-axis extending from the proximal end to the distal end of the sonotrode,
(b1) providing at least one stop member and attaching it to the ultrasound generating device, wherein the at least one stop member is isolated against ultrasound, wherein the at least one stop member at least partially or fully encloses the sonotrode around the y-axis, and wherein the at least one stop member is shorter than the sonotrode in y-axis direction from the proximal to the distal end of the sonotrode,
(c) providing at least one suture anchor having thermoplastic properties and attaching it to the distal end of the sonotrode, and
(d) providing an object with a recess, wherein the recess:
has a cross-section corresponding to or being larger than the cross-section of an implant, and
has a depth that is shorter than the length of the implant in y-direction, followed by
(f) inserting the suture anchor into the recess, followed by transmitting ultrasound from the sonotrode to the suture anchor for a time sufficient to liquify the suture anchor to the extent that liquification results in fixation of the suture by means of the anchor in the recess, and until the at least one stop member stops a movement of the device in y-axis direction by generating a mechanical resistance against a surface around the recess of the object.

6. A method for fixating a suture with a suture anchor in a recess of an object comprising the steps of:
(a) providing an ultrasound generating device having a sonotrode attached to a distal end of the ultrasound generating device at a proximal end of the sonotrode, wherein the sonotrode has a distal end configured to receive a suture anchor having thermoplastic properties and has a y-axis extending from the proximal end to the distal end of the sonotrode,
(b) providing at least one stop member and attaching it to the ultrasound generating device, wherein the at least one stop member is isolated against ultrasound, wherein the at least one stop member is shorter than the sonotrode in y-axis direction from the proximal to the distal end of the sonotrode,
(c) providing at least one suture anchor having thermoplastic properties and attaching it to the distal end of the sonotrode, and
(d) providing an object with a recess, wherein the recess:
has a cross-section corresponding to or being larger than the cross-section of an implant, and
has a depth that is shorter than the length of the implant in y-direction,
(e1) providing a sleeve having a distal and a proximal end,
(e2) positioning the sleeve with its distal end against a surface around the recess of the object, and
(e3) inserting the sonotrode in the sleeve,
followed by
(f) inserting the suture anchor into the recess, followed by transmitting ultrasound from the sonotrode to the suture anchor for a time sufficient to liquify the suture anchor to the extent that liquification results in fixation of the suture by means of the anchor in the recess, and until the at least one stop member stops a movement of the device in y-axis direction by generating a mechanical resistance against the proximal end of the sleeve.

7. The method according to claim 5, wherein after step (f), the method further comprises the following steps:
removing the at least one stop member of previous step (b), providing a second stop member and attaching it to the ultrasound generating device, wherein the second stop member is isolated against ultrasound, wherein the second stop member is shorter than the sonotrode in y-axis direction from the proximal to the distal end of the sonotrode, and wherein the second stop member is of a different length in y-axis direction compared to the at least one stop member of previous step (b), and
repeating steps (c) to (f).

8. The method according to claim 6, wherein after step (g), the method further comprises the following steps:
removing the at least one stop member of previous step (b), providing a second stop member and attaching it to the ultrasound generating device, wherein the second stop member is isolated against ultrasound, wherein the second stop member is shorter than the sonotrode in y-axis direction from the proximal to the distal end of the sonotrode, and wherein the second stop member is of a different length in y-axis direction compared to the at least one stop member of previous step (b), and
repeating steps (c) to (f).

9. The method according to claim 5, wherein after step (f), the method comprises the following steps:
providing a further suture anchor having thermoplastic properties and attaching it to the distal end of the sonotrode, wherein the further suture anchor is of a different size compared to the at least one suture anchor, and
repeating steps (d) to (f).

10. The method according to claim 6, wherein after step (f), the method comprises the following steps:
providing a further suture anchor having thermoplastic properties and attaching it to the distal end of the sonotrode, wherein the further suture anchor is of a different size compared to the at least one suture anchor, and
repeating steps (d) to (f).

11. The method according to claim 1, wherein the object is a human, animal or artificial hard tissue, a cartilage-covered hard tissue, or an implant structure.

12. The method according to claim 11, wherein at least one of:
the hard tissue is a hard tissue sample outside of a human or animal body, a hard tissue of a dead body or a hard tissue of a patient, and
the implant structure is a fixation plate or bone plate.

13. The method according to claim 1, wherein the sonotrode is a sonotrode comprising a body having
a proximal attached to the ultrasound generating device,
a distal end,
a tapered tip at the distal end of the body, wherein the tapered tip is tapered towards a distal end of the tip and wherein the tapered tip is configured to receive an implant having thermoplastic properties by inserting the tip at least partially into a cavity of the implant, and a z-axis from the distal end of the tapered tip to the proximal end of the body,
wherein an area of the tapered tip at the distal end of the body projected onto a plane normal to the z-axis fully covers an area of the distal end of the body projected onto the plane normal to the z-axis.

14. The method according to claim 1, wherein the stop member extends in direction of the distal end of the sonotrode along the y-axis.

15. The method according to claim 1, wherein the stop member at least partially or fully encloses the sonotrode around the y-axis.

16. The method according to claim 1, wherein the stop member is attached to the distal end of the ultrasound generating device.

17. The method according to claim 1, wherein a proximal end of the stop member is attached to the distal end of the ultrasound generating device, the stop member has a distal end, the stop member extends in direction of the distal end of the sonotrode along the y-axis, and wherein a distance between the distal end of the sonotrode and the distal end of the stop member along the y-axis is in a range of 1 mm to 10 mm.

18. The method according to claim 1, wherein the stop member forms a or forms part of a handpiece of the device.

19. The method according to claim 1, wherein the stop member forms a or forms part of a handpiece together with the ultrasound generating device.

20. The method according to claim 1, wherein the stop member is separably attached to the ultrasound generating device.

21. The method according to claim 1, wherein the device is configured to insert a pin or a suture anchor having thermoplastic properties in a recess of an object such that the pin or anchor sits flush with a surface around the recess of the object or such that there is a distance between a surface around the recess of the object and a proximal end of the pin or anchor of about 0.25 to 1.5 mm.

22. The method according to claim 1, wherein the device further comprises a sleeve slidably positioned around the sonotrode along the y-axis, wherein the sleeve has a distal and a proximal end, and is configured to stop a movement of the device in y-axis direction by generating a mechanical resistance at its distal end against a surface that is essentially perpendicular to the y-axis and at its proximal end against the stop member.

23. The method according to claim 10, wherein the sleeve has a slot from its distal end to its proximal end.

24. The method according to claim 10, wherein the sleeve has at least one recess at its proximal end or at its distal end.
